(19) United States
(12) Patent Application Publication
(10) Pub. No.: US 2023/0210374 A1
(43) Pub. Date: Jul. 6, 2023

(54) SYSTEMS, DEVICES, AND METHODS FOR WIRELESS ENERGY MANAGEMENT

(71) Applicant: uLink Labs, Inc., San Mateo, CA (US)

(72) Inventors: Jayant Charthad, Menlo Park, CA (US); Marcus Weber, San Francisco, CA (US)

(21) Appl. No.: 18/061,649

(22) Filed: Dec. 5, 2022

Related U.S. Application Data
(63) Continuation of application No. PCT/US2021/036258, filed on Jun. 7, 2021.
(60) Provisional application No. 63/036,302, filed on Jun. 8, 2020.

(51) Int. Cl. A61B 5/00

(52) U.S. Cl. CPC A61B 5/0031; A61B 5/6846; A61B 2560/0209

(57) ABSTRACT

Described herein are systems, devices, and methods for energy-efficient operation of wireless devices. In some variations, a wireless monitor may comprise a sensor configured to measure a physiological parameter of a patient at a first resolution. A processor may be configured to generate physiological parameter data based on the measured physiological parameter of the patient at the first resolution. The sensor may be configured to measure the physiological parameter of the patient at a second resolution based at least in part on the physiological parameter data.

### CROSS-REFERENCE TO RELATED APPLICATIONS

This application is a continuation of International Application No. PCT/US2021/036258, filed Jun. 7, 2021, which claims the benefit of U.S. Provisional Application No. 63/036,302, filed Jun. 8, 2020, each of which is incorporated by reference herein in its entirety.

### TECHNICAL FIELD

Devices, systems, and methods herein relate to energy-efficient operation of wireless devices including, but not limited to, wireless monitoring of one or more physiological parameters of a patient.

### BACKGROUND

Wireless monitors implanted inside a patient may monitor physiological signals (e.g., blood pressure, blood flow, neural action potentials, etc.) or stimulate tissue (e.g., nerve, muscle, etc.). These devices may receive wireless power provided by an external wireless device that may recharge a battery. An implanted wireless monitor may have limited space to accommodate the battery, which may limit the functionality and/or performance of the wireless monitor. As such, additional devices, systems, and methods may be desirable for one or more of energy-efficient operation of wireless devices and wireless monitoring of one or more physiological parameters of a patient.

### SUMMARY

Described herein are systems, devices, and methods for wireless energy management including, but not limited to, energy-efficient and reliable operation of wireless implantable devices and/or for monitoring a patient. In some variations, a wireless monitor may comprise a sensor configured to measure a physiological parameter of a patient at a first resolution, and a processor configured to generate physiological parameter data based on the measured physiological parameter of the patient at the first resolution, wherein the sensor may be configured to measure the physiological parameter of the patient at a second resolution based at least in part on the physiological parameter data.

In some variations, the first resolution may comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, a filter cut-off frequency, combinations thereof, and the like. In some variations, the second resolution may comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, a filter cut-off frequency, combinations thereof, and the like.

In some variations, measuring the physiological parameter at the first resolution may consume lower energy than measuring the physiological parameter at the second resolution. In some variations, the wireless monitor may be implanted in a patient. In some variations, the sensor may be configured to measure the physiological parameter of the patient at the first resolution periodically at a predetermined repetition interval. In some variations, the physiological parameter may comprise one or more of an intracardiac pressure, an intravascular pressure, a blood pressure, a blood velocity, a blood flow, a blood oxygen level, a heart rate, a breathing rate, a temperature, a voltage, a current, an impedance, a neural signal, a heart sound, combinations thereof, and the like.

In some variations, the measured physiological parameter at the first resolution and the second resolution may comprise digital bits. In some variations, the physiological parameter data may comprise digital bits. In some variations, the wireless monitor may further comprise a memory configured to store one or more of the measured physiological parameter at the first resolution, the measured physiological parameter at the second resolution, the physiological parameter data, resolution data, combinations thereof, and the like. In some variations, the wireless monitor may further comprise a wireless transmitter configured to wirelessly transmit one or more of the measured physiological parameter at the first resolution, the measured physiological parameter at the second resolution, the physiological parameter data, resolution data, combinations thereof, and the like, to a wireless device. In some variations, the wireless device may be an external wireless device configured to be disposed physically separate from the wireless monitor.

In some variations, the sensor may comprise one or more of a pressure transducer, a velocity sensor, a flow sensor, a blood oxygen sensor, a temperature sensor, an impedance sensor, an electrical sensor, a heart rate sensor, a breathing rate sensor, a neural sensor, an audio sensor, a front-end amplifier, a filter, an analog-to-digital converter, a comparator, a reference generator, a supply generator, a digital controller, a timer circuit, an oscillator, a clock circuit, combinations thereof, and the like.

In some variations, the sensor may comprise a resolution setting of the sensor. In some variations, the processor may be configured to adjust the resolution setting of the sensor based at least in part on the physiological parameter data. In some variations, the resolution setting may comprise one or more of a number of bits per sample, a voltage, a current, a sampling rate, a sampling duration, a number of samples, an over-sampling ratio (OSR), a frequency, a phase, an impedance, a filter cut-off frequency, combinations thereof, and the like. In some variations, the wireless monitor may further comprise a memory configured to store the resolution setting of the sensor. In some variations, the wireless monitor may further comprise a wireless transmitter configured to wirelessly transmit the resolution setting of the sensor to a wireless device. In some variations, the wireless device may be an external wireless device configured to be disposed physically separate from the wireless monitor.

Also described here are methods of monitoring a patient. In some variations, a method of monitoring a patient may comprise measuring a physiological parameter of the patient at a first resolution using a wireless monitor, generating physiological parameter data based on the measured physiological parameter at the first resolution, measuring the physiological parameter of the patient at a second resolution using the wireless monitor based at least in part on the physiological parameter data, and estimating a physiological state of the patient based at least in part on the measured physiological parameter at the second resolution.

In some variations, the first resolution may comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, a filter cut-off frequency, combinations thereof, and the like. In some variations, the second resolution may comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, a filter cut-off frequency, combinations thereof, and the like.

In some variations, measuring the physiological parameter at the first resolution may consume lower energy than measuring the physiological parameter at the second resolution. In some variations, the wireless monitor may be implanted in the patient. In some variations, the method may further comprise measuring the physiological parameter of the patient at the first resolution periodically at a predetermined repetition interval. In some variations, the physiological parameter may comprise one or more of an intracardiac pressure, an intravascular pressure, a blood pressure, a blood velocity, a blood flow, a blood oxygen level, a heart rate, a breathing rate, a temperature, a voltage, a current, an impedance, a neural signal, a heart sound, combinations thereof, and the like.

In some variations, generating physiological parameter data may comprise comparing the measured physiological parameter at the first resolution to a first threshold. In some variations, generating physiological parameter data may comprise calculating one or more of a mean, a median, a sum, a minimum, a maximum, combinations thereof, and the like, of a plurality of physiological parameter measurements at the first resolution. In some variations, generating physiological parameter data may comprise comparing one or more of the mean, the median, the sum, the minimum, the maximum, combinations thereof, and the like, of the plurality of physiological parameter measurements at the first resolution to a second threshold.

In some variations, the method may further comprise detecting a physiological event based at least in part on one or more of the measured physiological parameter at the first resolution and the physiological parameter data. In some variations, the detected physiological event may comprise one or more of arrhythmia, atrial fibrillation, ventricular tachycardia, sleep apnea, abnormally high blood pressure, abnormally low blood pressure, abnormal pressure variation, abnormal heart rate, abnormal heart rate variability, combinations thereof, and the like.

In some variations, the method may further comprise storing one or more of the measured physiological parameter at the first resolution, the measured physiological parameter at the second resolution, the physiological parameter data, resolution data, combinations thereof, and the like, in a memory of the wireless monitor. In some variations, the method may further comprise wirelessly transmitting one or more of the measured physiological parameter at the first resolution, the measured physiological parameter at the second resolution, the physiological parameter data, resolution data, combinations thereof, and the like, from the wireless monitor to a wireless device. In some variations, the wireless device may comprise an external wireless device configured to be disposed physically separate from the wireless monitor.

Also described here are wireless battery charging systems. In some variations, a wireless battery charging system may comprise a transducer configured to receive wireless power, a first power circuit coupled to the transducer and configured to recover at least a portion of the wireless power received by the transducer, a capacitor coupled to the first power circuit and configured to store at least a portion of the recovered wireless power from the first power circuit as capacitor energy, and a battery coupled to the capacitor and configured to charge using at least a portion of the capacitor energy during an absence of receiving the wireless power. In some variations, the battery may be configured to charge using at least another portion of the recovered wireless power from the first power circuit while receiving the wireless power.

In some variations, the wireless battery charging system may further comprise a second power circuit coupled to the capacitor and configured to condition at least the portion of the capacitor energy prior to charging the battery during the absence of receiving the wireless power. In some variations, the wireless battery charging system may further comprise a second power circuit coupled to the first power circuit and configured to condition at least the another portion of the recovered wireless power from the first power circuit prior to charging the battery while receiving the wireless power.

In some variations, the wireless battery charging system may further comprise a second power circuit coupled to the transducer and configured to recover at least another portion of the received wireless power from the transducer, wherein the battery may be configured to charge using at least a portion of the recovered wireless power from the second power circuit while receiving the wireless power.

In some variations, the wireless battery charging system may further comprise a third power circuit coupled to the first power circuit and configured to condition at least the portion of the recovered wireless power from the first power circuit prior to storing at least the portion of the recovered wireless power from the first power circuit in the capacitor as capacitor energy.

In some variations, the wireless battery charging system may further comprise at least a first switch coupled between the capacitor and the battery, wherein the at least first switch may be configured to be on during an absence of receiving the wireless power. In some variations, the wireless battery charging system may further comprise at least a second switch coupled between the second power circuit and the battery, wherein the at least second switch may be configured to be on while receiving the wireless power.

In some variations, the battery may have a capacity of less than about 100 milli-Watthour. In some variations, the capacitor may have capacitance between about 0.1 nF and about 100 μF. In some variations, the capacitor may store the recovered wireless power at a first rate, and the battery may charge using the capacitor energy at a second rate, wherein the first rate may be greater than the second rate.

In some variations, the first power circuit may comprise one or more of an AC-DC converter, a re-configurable AC-DC converter, a rectifier, a re-configurable rectifier, a DC-DC converter, a re-configurable DC-DC converter, a linear regulator, a switching regulator, a switched-capacitor voltage regulator, a voltage limiter, combinations thereof, and the like.

In some variations, the wireless battery charging system may further comprise a battery charging circuit coupled to the battery and configured to charge the battery. In some variations, the battery charging circuit may comprise one or more of a constant-voltage charging circuit, a constant-current charging circuit, a trickle charging circuit, a pulsed charging circuit, a DC-DC converter, a re-configurable DC-DC converter, a linear regulator, a switching regulator, a switched-capacitor voltage regulator, a voltage limiter configured to limit the voltage of the battery during charging, combinations thereof, and the like.

In some variations, the transducer may comprise an acoustic transducer, and the wireless power may comprise acoustic power. In some variations, the acoustic transducer may comprise an ultrasonic transducer, and the acoustic power may comprise ultrasonic power.

Also described here are methods of wirelessly charging a battery. In some variations, a method of wirelessly charging a battery may comprise receiving wireless power using a transducer, recovering at least a portion of the received wireless power using a first power circuit, storing at least a portion of the recovered wireless power from the first power circuit in a capacitor as capacitor energy, and charging the battery using at least a portion of the capacitor energy during an absence of receiving the wireless power. In some variations, the method may further comprise charging the battery using at least another portion of the recovered wireless power from the first power circuit while receiving the wireless power.

In some variations, the method may further comprise conditioning at least the portion of the capacitor energy using a second power circuit prior to charging the battery during the absence of receiving the wireless power. In some variations, the method may further comprise conditioning at least the another portion of the recovered wireless power from the first power circuit using a second power circuit prior to charging the battery while receiving the wireless power. In some variations, the method may further comprise recovering at least another portion of the received wireless power from the transducer using a second power circuit, and charging the battery using at least a portion of the recovered wireless power from the second power circuit while receiving the wireless power.

In some variations, the method may further comprise conditioning at least the portion of the recovered wireless power from the first power circuit using a third power circuit prior to storing at least the portion of the recovered wireless power from the first power circuit in the capacitor as capacitor energy.

In some variations, the battery may have a capacity of less than about 100 milli-Watthour. In some variations, the capacitor may have capacitance between about 0.1 nF and about 100 μF.

In some variations, the method may further comprise transmitting the wireless power to the transducer using a wireless device disposed physically separate from the transducer. In some variations, the transducer may comprise an acoustic transducer, and the wireless power may comprise acoustic power. In some variations, the acoustic transducer may comprise an ultrasonic transducer, and the acoustic power may comprise ultrasonic power.

Also described here are wireless implantable devices. In some variations, a wireless implantable device may comprise an energy storage device configured to provide power to the wireless implantable device, a load circuit coupled to the energy storage device and configured to perform a predetermined function, and a processor coupled to the energy storage device, the processor configured to measure an energy storage device parameter and generate a mode selection signal based at least in part on the measured energy storage device parameter, wherein the load circuit may be configured to perform the predetermined function in a first mode or a second mode based on the mode selection signal. In some variations, the energy storage device may comprise one or more of a battery and a capacitor.

In some variations, the device may further comprise a transducer configured to receive wireless power, a power circuit coupled to the transducer and configured to recover at least a portion of the received wireless power, and a power detector circuit coupled to the power circuit and the energy storage device, the power detector circuit configured to generate one or more supply voltages for one or more of the load circuit and the processor.

In some variations, the power detector circuit may comprise one or more of a power ORing circuit, a power combining circuit, a power selection circuit, a diode, and a switch. In some variations, the power detector circuit may be further configured to measure one or more of an energy storage device parameter and a power circuit parameter.

In some variations, the energy storage device parameter may comprise one or more of a battery voltage, a battery current, a battery impedance, a battery state of charge, a battery depth of discharge, a battery capacity, a battery energy, a battery power, a battery temperature, combinations thereof, and the like. In some variations, the energy storage device parameter may comprise one or more of a capacitor voltage, a capacitor current, a capacitor impedance, a capacitor state of charge, a capacitor depth of discharge, a capacitor energy, a capacitor temperature, combinations thereof, and the like. In some variations, the power circuit parameter may comprise one or more of a power circuit voltage, a power circuit current, a power circuit impedance, a power circuit power, a power circuit stored energy, a power circuit temperature, combinations thereof, and the like.

In some variations, the load circuit in the first mode may be configured to perform the predetermined function in an absence of a signal from an external device. In some variations, the load circuit in the second mode may be configured to perform the predetermined function in response to receiving a signal from an external device. In some variations, the predetermined function may comprise one or more of measuring a physiological parameter, measuring a parameter of the wireless implantable device, controlling the wireless implantable device, delivering stimulation, delivering therapy to a patient, combinations thereof, and the like. In some variations, the physiological parameter may comprise one or more of an intracardiac pressure, an intravascular pressure, a blood pressure, a blood velocity, a blood flow, a blood oxygen level, a heart rate, a breathing rate, a temperature, a voltage, a current, an impedance, a neural signal, a heart sound, combinations thereof, and the like.

In some variations, the energy storage device may have a capacity of less than about 100 milli-Watthour. In some variations, the device may further comprise a capacitor coupled to the power circuit, wherein the power circuit may be configured to store energy in the capacitor. In some variations, the processor may comprise one or more of an energy storage monitoring circuit, a battery monitoring circuit, a comparator, digital logic, combinations thereof, and the like. In some variations, the processor may be configured to be powered by one or more of the energy storage device and the power circuit.

In some variations, the transducer may comprise an acoustic transducer, and the wireless power may comprise acoustic power. In some variations, the acoustic transducer may comprise an ultrasonic transducer, and the acoustic power may comprise ultrasonic power.

Also described here are methods of operating a wireless implantable device. In some variations, a method of operating a wireless implantable device may comprise measuring an energy storage device parameter of an energy storage device of the wireless implantable device, generating a mode selection signal based on the measured energy storage device parameter, and configuring a load circuit to perform a predetermined function in a first mode or a second mode based on the mode selection signal. In some variations, the energy storage device may comprise one or more of a battery and a capacitor.

In some variations, the load circuit in the first mode may be configured to perform the predetermined function in an absence of a signal from an external device. In some variations, the load circuit in the second mode may be configured to perform the predetermined function in response to a signal from an external device. In some variations, the predetermined function may comprise one or more of measuring a physiological parameter, measuring a parameter of the wireless implantable device, controlling the wireless implantable device, delivering stimulation, delivering therapy to a patient, combinations thereof, and the like. In some variations, the physiological parameter may comprise one or more of an intracardiac pressure, an intravascular pressure, a blood pressure, a blood velocity, a blood flow, a blood oxygen level, a heart rate, a breathing rate, a temperature, a voltage, a current, an impedance, a neural signal, a heart sound, combinations thereof, and the like. In some variations, the energy storage device may have a capacity of less than about 100 milli-Watthour.

In some variations, the method may further comprise receiving wireless power using a transducer of the wireless implantable device, recovering at least a portion of the received wireless power using a power circuit of the wireless implantable device, and generating one or more supply voltages for the load circuit using a power detector circuit of the wireless implantable device.

In some variations, the method may further comprise measuring one or more of an energy storage device parameter and a power circuit parameter using the power detector circuit. In some variations, the energy storage device parameter may comprise one or more of a battery voltage, a battery current, a battery impedance, a battery state of charge, a battery depth of discharge, a battery capacity, a battery energy, a battery power, a battery temperature, combinations thereof, and the like. In some variations, the energy storage device parameter may comprise one or more of a capacitor voltage, a capacitor current, a capacitor impedance, a capacitor state of charge, a capacitor depth of discharge, a capacitor energy, a capacitor temperature, combinations thereof, and the like. In some variations, the power circuit parameter may comprise one or more of a power circuit voltage, a power circuit current, a power circuit impedance, a power circuit power, a power circuit stored energy, combinations thereof, and the like.

In some variations, the method may further comprise storing energy in a capacitor of the wireless implantable device using the power circuit. In some variations, the power detector circuit may comprise one or more of a power ORing circuit, a power combining circuit, a power selection circuit, a diode, a switch, combinations thereof, and the like. In some variations, the transducer may comprise an acoustic transducer, and the wireless power may comprise acoustic power. In some variations, the acoustic transducer may comprise an ultrasonic transducer, and the acoustic power may comprise ultrasonic power.

Also described here are wireless implantable devices. In some variations, a wireless implantable device may comprise a transducer configured to receive a wireless signal, a wakeup receiver circuit coupled to the transducer, the wakeup receiver circuit configured to generate a wakeup signal in response to the wireless signal, a processor configured to generate a trigger signal for the wakeup receiver circuit based on a timer signal, and an energy storage device configured to provide power to the wakeup receiver circuit and the processor, wherein the wakeup receiver circuit may be configured to operate only upon receiving the trigger signal.

In some variations, the energy storage device may comprise one or more of a battery and a capacitor. In some variations, the battery may have a capacity of less than about 100 milli-Watthour.

In some variations, the processor may comprise a timer circuit configured to generate the timer signal. In some variations, the trigger signal may comprise a periodic waveform having a predetermined repetition interval. In some variations, the predetermined repetition interval may be less than or equal to a duration of the wireless signal received by the transducer.

In some variations, the device may further comprise a power circuit coupled to the energy storage device, the power circuit configured to generate one or more supply voltages for powering the wakeup receiver circuit and the processor.

In some variations, the wireless signal may be transmitted by a wireless device configured to be disposed physically separate from the wireless implantable device. In some variations, the transducer may comprise an acoustic transducer, and the wireless signal may comprise an acoustic signal. In some variations, the acoustic transducer may comprise an ultrasonic transducer, and the acoustic signal may comprise an ultrasonic signal.

In some variations, an implantable device may comprise an energy storage device configured to provide power to the implantable device, an energy storage monitoring circuit configured to monitor one or more energy storage device parameters, and a processor configured to generate a trigger signal for the energy storage monitoring circuit based on the monitoring of the one or more energy storage device parameters, wherein the energy storage monitoring circuit may be configured to operate only upon receiving the trigger signal.

In some variations, the energy storage device may comprise one or more of a battery and a capacitor. In some variations, the battery may have a capacity of less than about 100 milli-Watthour. In some variations, the processor may comprise a timer circuit. In some variations, the trigger signal may comprise a periodic waveform having a predetermined repetition interval.

In some variations, the one or more energy storage device parameters may comprise one or more of a battery voltage, a battery current, a battery impedance, a battery state of charge, a battery depth of discharge, a battery capacity, a battery energy, a battery power, a battery temperature, combinations thereof, and the like. In some variations, the one or more energy storage device parameters comprise one or more of a capacitor voltage, a capacitor current, a capacitor impedance, a capacitor state of charge, a capacitor depth of discharge, a capacitor energy, a capacitor temperature, combinations thereof, and the like.

In some variations, the processor may be further configured to estimate one or more energy storage device parameters. In some variations, the processor may be configured to generate the trigger signal based at least in part on the estimation of the one or more energy storage device parameters.

In some variations, the device may further comprise a power circuit coupled to the energy storage device, the power circuit configured to generate one or more supply voltages for powering the energy storage monitoring circuit and the processor.

Also described here are methods of cardiovascular pressure monitoring. In some variations, a method of cardiovascular pressure monitoring may comprise measuring cardiovascular pressure using a wireless monitor implanted in a patient, measuring at least one other physiological parameter of the patient, and determining a patient status based at least in part on the cardiovascular pressure and the at least one other physiological parameter of the patient.

In some variations, the method may further comprise synchronizing the measured cardiovascular pressure with the measurement of the other physiological parameter. In some variations, synchronization may be performed by an external device configured to be disposed physically separate from the wireless monitor.

In some variations, the other physiological parameter may comprise one or more of a patient activity, a heart rate, a heart rate variability, a breathing rate, a thoracic impedance, a heart sound, a temperature, a blood pressure, a blood flow, a blood velocity, a blood oxygen level, a blood glucose level, combinations thereof, and the like.

In some variations, measuring the at least one other physiological parameter of the patient may be performed by an external device configured to be disposed physically separate from the wireless monitor. In some variations, measuring the at least one other physiological parameter of the patient may be performed using a second wireless monitor implanted in the patient. In some variations, measuring the at least one other physiological parameter of the patient may be performed using the wireless monitor implanted in the patient.

In some variations, the method may further comprise digitizing the measured cardiovascular pressure using a processor of the wireless monitor. In some variations, the method may further comprise wirelessly transmitting digitized cardiovascular pressure from the wireless monitor to a wireless device. In some variations, the wireless device may comprise an external wireless device configured to be disposed physically separate from the wireless monitor. In some variations, the wireless transmission may be performed using one or more of an acoustic signal, an ultrasonic signal, and a radio-frequency signal.

### BRIEF DESCRIPTION OF THE DRAWINGS

FIG. 1 is a schematic block diagram of an illustrative variation of a wireless implantable device.

FIG. 2 is an illustrative flowchart of a variation of a method of monitoring a patient.

FIG. 3 is a timing diagram of an illustrative variation of a method of monitoring a patient.

FIG. 4 is a schematic block diagram of an illustrative variation of a wireless monitor comprising a sensor and a processor.

FIG. 5 is an illustrative flowchart of a variation of a method of wirelessly charging an energy storage device.

FIGS. 6A and 6B are schematic block diagrams of illustrative variations of a wireless battery charging system comprising a first power circuit.

FIGS. 7A and 7B are schematic block diagrams of illustrative variations of a wireless battery charging system comprising a first power circuit and a second power circuit.

FIGS. 8A and 8B are schematic block diagrams of illustrative variations of yet another wireless battery charging system comprising a first power circuit and a second power circuit.

FIGS. 9A and 9B are schematic block diagrams of illustrative variations of a wireless battery charging system comprising a first power circuit, a second power circuit, and a third power circuit.

FIG. 10 is a timing diagram of an illustrative variation of a method of wirelessly charging a battery.

FIG. 11 is an illustrative flowchart of a variation of a method of operating a wireless implantable device.

FIG. 12 is a schematic block diagram of an illustrative variation of a wireless implantable device comprising an energy storage device, a load circuit, and a processor.

FIGS. 13A and 13B are schematic block diagrams of illustrative variations of a power detector circuit.

FIG. 14 is a schematic block diagram of an illustrative variation of a wireless implantable device comprising a wakeup receiver circuit.

FIG. 15 is an illustrative flowchart of a variation of a method of operating an energy storage monitoring circuit.

FIG. 16 is a schematic block diagram of an illustrative variation of an implantable device comprising an energy storage monitoring circuit.

FIG. 17 is an illustrative flowchart of a variation of a method of cardiovascular pressure monitoring.

### DETAILED DESCRIPTION

### I. Systems

A. Overview

Generally described herein are systems, devices, and methods for wireless energy management including, but not limited to, energy-efficient and reliable operation of wireless implantable devices and/or for monitoring a patient. Generally, a wireless system may comprise one or more wireless monitors or wireless implantable devices, and one or more wireless devices or external wireless devices. The wireless monitor may be implanted in a patient's body for performing one or more functions such as monitoring physiological signals (e.g., blood pressure, blood flow, neural action potentials, etc.) or stimulating tissue (e.g., nerve, muscle, etc.). The wireless monitor may perform its functions using wireless power provided by an external wireless device, or it may comprise an energy storage device (e.g., capacitor, battery, etc.), which may be recharged by the wireless device using wireless power transfer. The wireless monitor may also wirelessly communicate data and/or commands bi-directionally with the wireless device.

Due to size constraints, operating the wireless monitor or the wireless implantable device to perform a function (e.g., monitoring a physiological parameter of patient) while consuming minimal amount of energy (e.g., to prolong the battery life of the device) may be challenging. In such systems, reducing the energy consumption of the wireless monitor may, for example, prolong the battery life of an implantable device.

Furthermore, wirelessly charging an energy storage device of an implantable device with high efficiency or in a short time may also be challenging due to potential interruptions in the wireless link, and/or due to limited charging rates of miniature batteries. Therefore, efficiently recharging an energy storage device of the wireless monitor may shorten the time required for wireless charging and improve usability for the patient.

In some variations, energy-efficient monitoring using a wireless monitor may be facilitated by using a plurality of sensing resolutions. For example, a wireless monitor may comprise a sensor configured to measure a physiological parameter of a patient at a first resolution, and a processor configured to generate physiological parameter data based on the measured physiological parameter of the patient at the first resolution. The sensor may be configured to measure the physiological parameter of the patient at a second resolution based at least in part on the physiological parameter data.

In some variations, wireless charging of an energy storage device may utilize a battery and capacitor. For example, a wireless battery charging system may comprise a transducer configured to receive wireless power, a first power circuit coupled to the transducer and configured to recover at least a portion of the wireless power received by the transducer, a capacitor coupled to the first power circuit and configured to store at least a portion of the recovered wireless power from the first power circuit as capacitor energy, and a battery coupled to the capacitor and configured to charge using at least a portion of the capacitor energy during an absence of receiving the wireless power.

In some variations, a wireless implantable device may be operated in a plurality of modes. For example, a wireless implantable device may comprise an energy storage device configured to provide power to the wireless implantable device, a load circuit coupled to the energy storage device and configured to perform a predetermined function, and a processor coupled to the energy storage device. The processor may be configured to measure an energy storage device parameter and generate a mode selection signal based at least in part on the measured energy storage device parameter. The load circuit may be configured to perform the predetermined function in a first mode or a second mode based on the mode selection signal.

In some variations, a wireless implantable device may be duty-cycled to increase energy efficiency. For example, a wireless implantable device may comprise a transducer configured to receive a wireless signal, a wakeup receiver circuit coupled to the transducer, the wakeup receiver circuit configured to generate a wakeup signal in response to the wireless signal, a processor configured to generate a trigger signal for the wakeup receiver circuit based on a timer signal, and an energy storage device configured to provide power to the wakeup receiver circuit and the processor. The wakeup receiver circuit may be configured to operate only upon receiving the trigger signal.

B. Wireless Monitor

Generally, a wireless monitor may be configured to perform one or more functions including, but not limited to, sensing, monitoring, stimulation, delivering therapy, combinations thereof, and the like. In some variations, the wireless monitor may receive and/or transmit one or more of wireless power, wireless data, wireless commands, and wireless signals to/from an external wireless device or another wireless monitor. For example, the wireless monitor may be configured to monitor, measure and/or process one or more physiological parameters of a patient.

In some variations, the wireless monitors described herein may be configured to perform only a predetermined sub-set of the measurements, processing, data storage, and/or signal transmission steps described herein. In some variations, the wireless monitors may comprise only a sub-set of the components or blocks described herein. For example, in some variations, a wireless monitor may include only a transducer, a power circuit and a processor. As another example, in some variations, a wireless monitor may include one or more transducers, a power circuit, a processor, a sensor and a memory. In some variations, a wireless monitor may comprise other components in addition to what may be described herein (e.g., sensors, stimulators, delivery and/or anchoring mechanisms, mechanical parts to enable deployment in the body or organ, or other components).

In some variations, a wireless monitor may be implanted inside a patient or an animal. In some variations, a wireless monitor, as described herein, may be coupled (e.g., attached) to an implantable device, or any part of an implantable device. For example, one or more wireless monitors may be attached to a prosthetic heart valve or a stent. As another example, one or more wireless monitors may be attached to one or more of a pulse generator and/or one or more leads of a pacemaker, an implantable cardioverter defibrillator, and/or cardiac resynchronization therapy devices. In some variations, the wireless monitor may be implanted within or on one or more of a cardiac structure (e.g., heart valve, heart chamber), a vascular structure (e.g., pulmonary artery, any other blood vessel), body lumen, body cavity, tissue, organ, and the like.

In some variations, a wireless monitor may comprise one or more components or blocks described herein for an implantable device. For example, a wireless monitor may comprise one or more of a transducer, a power circuit, an energy storage device, a load circuit, a sensor, a processor, a memory, a wireless transmitter, a wakeup receiver circuit, a multiplexer circuit, combinations thereof, and the like.

C. Implantable Device

Generally, an implantable device described herein may be configured to be implanted inside a patient or an animal. In some variations, the implantable device may be a wireless implantable device. In some variations, the wireless implantable device may receive and/or transmit one or more of wireless power, wireless data, wireless commands, and wireless signals to/from an external wireless device or another wireless implantable device. In some variations, a wireless implantable device may be configured to perform one or more functions including, but not limited to, sensing, monitoring, stimulation, delivering therapy, combinations thereof, and the like. In some variations, a wireless implantable device may be a wireless monitor.

In some variations, an implantable device may comprise one or more of a prosthetic heart valve, prosthetic heart valve conduit, valve leaflet coaptation devices, annuloplasty rings, valve repair devices (e.g., clips, pledgets), septal occluders, appendage occluders, ventricular assist devices, pacemakers (e.g., including leads, pulse generator), implantable cardioverter defibrillators (e.g., including leads, pulse generator), cardiac resynchronization therapy devices (e.g., including leads, pulse generator), insertable cardiac monitors, stents (e.g., coronary or peripheral stents, fabric stents, metal stents), stent grafts, scaffolds, embolic protection devices, embolization coils, endovascular plugs, vascular patches, vascular closure devices, interatrial shunts, parachute devices for treating heart failure, cardiac loop recorders, combinations thereof, and the like. For example, a prosthetic heart valve may comprise one or more of a transcatheter heart valve (THV), self-expandable THV, balloon expandable THV, surgical bioprosthetic heart valve, mechanical valve, and the like.

Generally, the implantable devices described herein may be located in or near (e.g., adjacent, proximal) any region in the body including, but not limited to, a heart valve (e.g., aortic valve, mitral valve), a heart chamber (e.g., left ventricle or LV, left atrium or LA, right ventricle or RV, right atrium or RA), a blood vessel (e.g., pulmonary artery, aorta, superficial femoral artery, coronary artery, pulmonary vein, and the like), heart tissue (e.g., heart muscle or wall, septum), gastrointestinal tract (e.g., stomach, esophagus), bladder, combinations thereof, and the like.

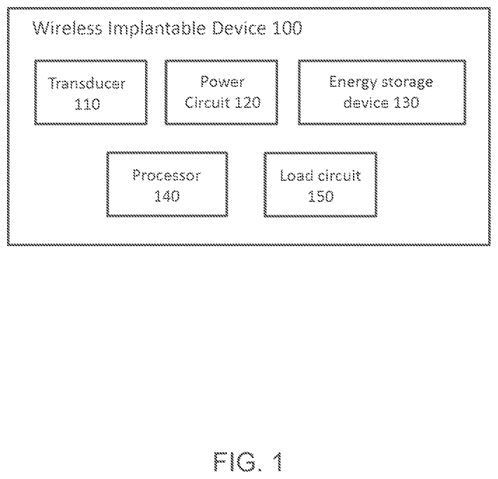

FIG. 1 is a schematic block diagram of a wireless implantable device (100) comprising a transducer (110), a power circuit (120), an energy storage device (130), a processor (140), and a load circuit (150), where each of the components are described in more detail herein.

a. Transducer

Generally, a transducer described herein may be configured to convert between a wireless energy modality and electrical signals. In some variations, a transducer of a device may be configured to exchange one or more of wireless power, a wireless signal, wireless data, a wireless command, combinations thereof, and the like, with another device and/or with another transducer of the same device. In some variations, the transducer (110) may be configured to receive and/or transmit signals using one or more of mechanical waves (e.g., acoustic, ultrasonic or ultrasound, vibrational), magnetic fields (e.g., inductive), electric fields (e.g., capacitive), electromagnetic waves (e.g., radiofrequency or RF, optical), galvanic coupling, surface waves, combinations thereof, and the like, as well as convert the signals into and/or from electrical signals. A transducer, as described herein, may be included in one or more of a wireless implantable device, a wireless monitor, an external wireless device, and the like (e.g., any of the devices described herein).

In some variations, a transducer (110) may comprise one or more of an ultrasonic transducer, a radiofrequency (RF) transducer (e.g., a coil, an RF antenna), a capacitive transducer, combinations thereof, and the like. In some variations, an ultrasonic transducer may comprise one or more of a piezoelectric device, a capacitive micromachined ultrasonic transducer (CMUT), a piezoelectric micromachined ultrasonic transducer (PMUT), combinations thereof, and the like. In some variations, an ultrasonic transducer may convert pressure and/or force into an electrical signal, and/or vice versa. In some variations, the transducer (110) may comprise one or more ultrasonic transducers that may be of one or more types, including but not limited to, piston (e.g., rod, plate), cylindrical, ring, spherical (e.g., shell), flexural (e.g., bar, diaphragm), flextensional, combinations thereof, and the like. In some variations, a piezoelectric device may be made of one or more of lead zirconate titanate (PZT), PMN-PT, Barium titanate (BaTiO3), polyvinylidene difluoride (PVDF), Lithium niobate (LiNbO3), any derivates thereof, and the like. In some variations, a radiofrequency (RF) transducer may be configured for transmitting and/or receiving near-field and/or non-near-field (e.g., far-field) signals. For example, an RF antenna may be configured for non-near-field transmission and/or reception of power, data and/or other signals. An RF coil may be configured for near-field (e.g., inductive) transmission and/or reception of power, data and/or other signals.

In some variations, a transducer (110) may comprise one or more ultrasonic transducers for one or more of receiving wireless power, transmitting/receiving data to/from another wireless device, and transmitting/receiving signals to/from another wireless device. For example, an ultrasonic transducer of a wireless monitor may be designed to operate at a frequency between about 20 kHz and about 20 MHz for receiving power from an external wireless device. Operation in such a frequency range may be useful to miniaturize an ultrasonic transducer to millimeter or sub-millimeter dimensions, which may be advantageous for integrating one or more wireless monitors onto another implantable device (e.g., a transcatheter heart valve, a stent). In some variations, an ultrasonic transducer may have an impedance with a real part in the order of about hundreds of Ohms to about hundreds of kilo Ohms (e.g., between about 100Ω and about 500 kΩ). In some variations, an ultrasonic transducer may have an impedance with a real part in the order of tens of Ohms.

In some variations, a transducer (110) may comprise a single transducer element (e.g., ultrasonic piezoelectric device) that may allow miniaturization of the wireless monitor. In some variations, the single transducer element may be configured to receive a power signal (e.g., ultrasonic power) transmitted from an external wireless device and convert the signal to electrical power. Additionally, or alternatively, the single transducer element may be configured to receive downlink data (e.g., using an ultrasonic signal) and/or other signals from an external wireless device or a wireless monitor. In some variations, the single transducer element may be configured to transmit uplink data (e.g., using an ultrasonic signal) and/or other signals to an external wireless device or a wireless monitor. In some variations, the single transducer element may comprise an ultrasonic transducer configured to perform one or more of receiving ultrasonic power from another device (e.g., external wireless device), performing bi-directional ultrasonic data communication or signal exchange (e.g., uplink and downlink) with another device (e.g., external wireless device, wireless monitor), combinations thereof, and the like.

In some variations, a transducer (110) may comprise more than one transducer element or one or more arrays of transducer elements. For example, the transducer (110) may comprise an array of ultrasonic transducer elements. As another example, a first transducer element may comprise an RF coil configured to receive power and communicate data and/or other signals with an external wireless device. A second transducer element may comprise an ultrasonic transducer configured to transmit and/or receive other signals. In some variations, an ultrasonic transducer of an external wireless device may comprise one or more arrays of ultrasonic transducer elements configured to generate an ultrasonic beam for one or more of power transfer, data transfer and/or exchange of other signals with a wireless monitor.

In some variations, a transducer (110) comprising a plurality of transducer elements may be configured to perform a predetermined set of functions. For example, a first transducer element may be configured to recover wireless power, a second transducer element may be configured to receive data and/or signals, and a third transducer element may be configured to transmit data and/or signals.

Small transducer size may allow one or more wireless monitors to be miniaturized, which may be useful for attaching one or more wireless monitors to another implantable device such as a cardiac implantable device (e.g., prosthetic heart valve), and/or may allow minimally invasive delivery of the wireless monitor or wireless implantable device into the body (e.g., via percutaneous or transcatheter techniques). In some variations, a transducer may have a volume of less than about 10 cm³.

In some variations, a transducer (e.g., an ultrasonic transducer) of a wireless monitor may be oriented or angled towards one or more of a transducer of another wireless monitor, a transducer of the external wireless device, combinations thereof, and the like. This may facilitate the reliability of transmitting/receiving power, data and/or other signals between a wireless monitor and an external wireless device, or between two wireless monitors.

In some variations, a wireless monitor may comprise one or more transducers. In some variations, one or more wireless monitors may share one or more transducers. For example, in some variations, more than one wireless monitor may be connected to a transducer (e.g., an RF coil) with more than one feed or port. For example, a stent device may comprise an RF coil with two or more feeds or ports, to which two or more wireless monitors may be connected. In some variations, two or more wireless monitors may be connected to a single feed or port of a transducer (e.g., two or more wireless monitors connected in parallel at a single feed or port of an RF coil).

b. Power Circuit

Generally, a power circuit described herein may be configured to recover, condition, detect, select, combine, store and/or supply power or energy. For example, a power circuit may be configured to recover wireless power received by a transducer and convert it into usable energy for powering one or more circuit blocks of a wireless monitor. In some variations, the power circuit may comprise one or more energy storage elements (e.g., battery, capacitor) configured to store energy received by the transducer. The power circuit may be further configured to control (e.g., regulate, limit) the power provided to one or more components (e.g., circuit blocks) of the wireless monitor. The combination of the power circuits and transducers described herein may be useful for power, data and/or signal transfer between an external wireless device and one or more low-power devices (e.g., wireless monitor) implanted in a patient. In some variations, the power circuit (120) may comprise one or more of a power recovery circuit, a power management circuit, a power detector circuit, a power distribution circuit, combinations thereof, and the like.

In some variations, the power circuit (120) may comprise an AC-DC converter configured to convert alternating current (AC) voltage into a DC voltage. For example, the power circuit (120) may comprise a rectifier configured to convert AC voltage at the terminals of a transducer into a DC voltage rail. The rectifier may comprise one or more of a passive rectifier, an active rectifier, a passive voltage doubler, combinations thereof, and the like. In some variations, the power circuit (120) may comprise a DC-DC converter configured to convert a DC voltage rail into another DC voltage rail. For example, the power circuit (120) may comprise a switched-capacitor DC-DC converter, a charge pump, combinations thereof, and the like. In some variations, the power circuit (120) may comprise a voltage regulator (e.g., a low-dropout regulator (LDO) circuit, a voltage clamp circuit) configured to generate a regulated or constant DC voltage rail. In some variations, the power circuit (120) may comprise one or more reference generation circuits such as a current reference circuit, a bandgap reference circuit, a voltage reference circuit, combinations thereof, and the like.

In some variations, the power circuit (120) may be configured to recover and/or combine wireless power received by a plurality of transducer elements located on a wireless monitor. For instance, such a power circuit connected to a plurality of transducer elements may perform one or more of AC power combining, DC power combining, DC voltage combining, DC current combining, any combinations thereof, and the like.

In some variations, the power circuit (120) may comprise a power detector circuit configured to detect or measure power and/or energy at one or more of its inputs. In some variations, the power detector circuit may be configured to provide one or more supply voltages or power to one or more circuit blocks in a wireless monitor depending on detection of power at one or more inputs. In some variations, the power detector circuit may comprise one or more of a power ORing circuit, a power combining circuit, a power selection circuit, one or more diodes and one or more switches, as described herein. A power ORing circuit, a power combining circuit or a power selection circuit may generally operate on a plurality of power sources at its input and generate one or more power or voltage supplies at its output. For example, a power combining circuit may combine power from a plurality of sources. For example, a power selection circuit may select power from a power source out of a plurality of power sources.

In some variations, the power circuit (120) may comprise an energy storage device (130) comprising one or more of a capacitor, a super-capacitor, a rechargeable or secondary battery, a non-rechargeable or primary battery, combinations thereof, and the like. In some variations, the power circuit (120) may comprise a rechargeable battery for energy storage, along with a capacitor in parallel with the battery, wherein the capacitor may sink/supply at least a part of the current during charging/discharging transients of the rechargeable battery.

In some variations, the power circuit (120) may be separate from an energy storage device (130). In some variations, the power circuit (120) may not include any energy storage device, and the wireless monitor may be powered by another device (e.g., external wireless device, another wireless monitor, and the like) during the operation of the wireless monitor. In some variations, power may be provided to a wireless monitor until it completes a predetermined set of functions, and the wireless monitor may remain inactive until it is powered again. A power circuit without an energy storage device may allow reduction in the size of the power circuit and the wireless monitor.

In some variations, the systems, devices, and methods disclosed herein may comprise one or more systems, devices, and methods described in U.S. Pat. No. 9,544,068, filed on May 13, 2014, U.S. Pat. No. 10,177,606, filed on Sep. 30, 2016, and U.S. Pat. No. 10,014,570, filed on Dec. 7, 2016, the contents of each of which are hereby incorporated by reference in its entirety.

c. Energy Storage Device

Generally, an energy storage device described herein may be configured to store energy, which may be used to power one or more circuit blocks of a wireless implantable device or wireless monitor. In some variations, an energy storage device (130) may comprise one or more of a capacitor, a super-capacitor, a rechargeable or secondary battery, a non-rechargeable or primary battery, combinations thereof, and the like.

In some variations, an energy storage device (130) of a wireless implantable device (100) may comprise a battery (e.g., a rechargeable battery) with a capacity of less than about 100 milli-Watthour (about 360 Joules). In some variations, an energy storage device (130) of a wireless implantable device (100) may comprise a battery (e.g., a rechargeable battery) with a capacity of less than about 10 milli-Watthour (36 Joules). Such a battery may be significantly smaller in size than batteries used in conventional implantable devices such as pacemakers or deep brain stimulators, allowing miniaturization of the wireless implantable device (100) to dimensions on the order of a centimeter, a millimeter, or less than a millimeter.

In some variations, an energy storage device (130) of a wireless implantable device (100) may comprise a capacitor with capacitance between about 0.1 nano-Farads (nF) and about 100 micro-Farads (μF). Such a capacitor may be on-chip (i.e., included within an integrated circuit) or off-chip. In some variations, a wireless implantable device (100) may comprise a plurality of energy storage devices (130), each of which may comprise any type of energy storage device described herein.

d. Load Circuit

Generally, a load circuit (150) of a wireless implantable device (100) described herein may be configured to perform a predetermined function. In some variations, the predetermined function may comprise one or more of measuring a physiological parameter (e.g., intracardiac pressure, heart rate, and the like), measuring a parameter of the wireless implantable device (e.g., a structural parameter of the implantable device such as the thickness or motion of a prosthetic heart valve leaflet, tissue growth around the implantable device, scar tissue, and the like), controlling the wireless implantable device, delivering stimulation (e.g., electrical stimulation to a nerve, muscle or other tissue), and delivering therapy (e.g., drug delivery) to a patient. In some variations, a load circuit (150) may comprise a sensor or a sensing circuit (e.g., a pressure sensor or pressure sensing circuit), a diagnostic circuit, a monitoring circuit (e.g., to monitor pressure), a controller, a stimulation circuit (e.g., for stimulating a nerve, muscle or other tissue), a therapy circuit, combinations thereof, and the like. For example, in some variations, a sensor or a sensing circuit may be configured to measure a physiological parameter (e.g., cardiovascular pressure) of a patient. In some variations, a stimulation circuit may be configured to apply electrical stimulation to nerve tissue.

In some variations, a load circuit (150) may be configured to operate (e.g., perform a predetermined function) in a plurality of modes. For example, a load circuit (150) may be configured to perform a predetermined function in a first mode or a second mode based on a mode selection signal generated by a processor (140) as described herein.

e. Sensor

Generally, a sensor described herein may be configured to sense or measure one or more parameters. In some variations, the sensor may comprise one or more of a pressure sensor, a flow sensor, a transducer (e.g., an ultrasonic transducer, an infrared/optical photodiode, an infrared/optical LED, an RF antenna, an RF coil), a temperature sensor, an electrical sensor (e.g., using electrodes for measuring impedance, electromyogram or EMG, electrocardiogram or ECG, and the like), a magnetic sensor (e.g., RF coil), an electromagnetic sensor (e.g., infrared photodiode, optical photodiode, RF antenna), a neural sensor (e.g., for sensing neural action potentials), a force sensor (e.g., a strain gauge), a flow or a velocity sensor (e.g., hot wire anemometer, vortex flowmeter), an acceleration sensor (e.g., accelerometer), a chemical sensor (e.g., pH sensors, protein sensor, glucose sensor), an oxygen sensor (e.g., pulse oximetry sensor, myocardial oxygen consumption sensor), an audio sensor (e.g., a microphone to detect heart murmurs, prosthetic valve murmurs, auscultation), a sensor for sensing other physiological parameters (e.g., sensors to sense heart rate, breathing rate, arrhythmia, motion of heart walls), a stimulator (e.g., for stimulation and/or pacing function), combinations thereof, and the like.

In some variations, one or more pressure sensors (alternatively referred to as a pressure transducer) may be used for one or more of monitoring heart function and/or heart failure (e.g., measuring pressure in the LV, RV, LA, RA, pulmonary artery, aorta, and the like), monitoring a prosthetic valve (e.g., valve pressure gradients to monitor stenosis), monitoring a stent device (e.g., measuring pressure in the lumen), estimation and/or verification of blood velocity measurements (e.g., using the Bernoulli equation), combinations thereof, and the like. In some variations, one or more pressure sensors may be of the following types including, but not limited to, an absolute pressure sensor, a gauge pressure sensor, a sealed pressure sensor, a differential pressure sensor, an atmospheric pressure sensor, combinations thereof, and the like. In some variations, one or more pressure sensors may be based upon one or more pressure-sensing technologies including, but not limited to, resistive (e.g., piezoresistive, using a strain gauge or a membrane to create a pressure-sensitive resistance, and the like), capacitive (e.g., using a diaphragm or a membrane to create a pressure-sensitive capacitance, and the like), piezoelectric, optical, resonant (e.g., pressure-sensitive resonance frequency of a structure, and the like), combinations thereof, and the like. In some variations, a pressure sensor may be manufactured using Micro-Electro-Mechanical Systems (MEMS) technology. In some variations, a pressure sensor may comprise one or more of a stagnation pressure sensor, a static pressure sensor, and the like.

In some variations, a sensor may comprise a stimulator used for stimulating muscles and/or neurons or nerves of one or more of cardiac tissue (e.g., HIS bundle, atrioventricular node), heart chamber (e.g., septal, lateral walls of the LV), blood vessel wall, combinations thereof, and the like. For example, one or more stimulators may be used to stimulate the LV wall for pacing and/or cardiac resynchronization. In some variations, a stimulator may comprise an electrical stimulator (e.g., electrodes), an ultrasonic stimulator (e.g., ultrasonic transducer), an optical stimulator (e.g., an optical LED), an infrared stimulator (e.g., an infrared LED), a thermal stimulator (e.g., electrodes to generate heat in tissue), combinations thereof, and the like.

In some variations, a sensor may comprise one or more of a sensing transducer and sensing circuits. In some variations, sensing circuits may comprise one or more of a signal conditioning circuit, an analog front-end (AFE), an amplifier, front-end amplifier (FEA), an instrumentation amplifier, a filter, an anti-aliasing filter, an analog-to-digital converter (ADC), a comparator, a reference generator, a supply generator, a digital controller, a bias circuit, a clock circuit, a timer circuit, an oscillator, combinations thereof, and the like.

In some variations, a sensor may be configured to measure a physiological parameter of a patient. In some variations, the physiological parameter of the patient may comprise one or more of an intracardiac pressure, an intravascular pressure, a blood pressure, a blood velocity, a blood flow, a blood oxygen level, a heart rate, a breathing rate, a temperature, a voltage (e.g., an electrical voltage generated by tissue such as ECG, EMG, and the like), a current, an impedance (e.g., tissue impedance, thoracic impedance, and the like), a neural signal, a heart sound, combinations thereof, and the like.

In some variations, a sensor may be configured to measure one or more physiological parameters at one or more resolutions. For example, a sensor may be configured to measure a physiological parameter at a first resolution and/or at a second resolution. Resolution may refer to one or more of a resolution in the amplitude of the sensed parameter, and a resolution in the timing of the sensed parameter. In some variations, the first and/or the second resolution may comprise one or more of an amplitude of a physiological parameter (e.g., pressure resolution), a timing of a physiological parameter, number of bits per sample, a voltage, a current, a sampling rate, a sampling duration, a number of samples, an over-sampling ratio (OSR), a frequency, a phase, an impedance, a filter cut-off frequency, combinations thereof, and the like.

In some variations, a sensor may comprise one or more resolution settings of the sensor. In some variations, a resolution setting may control a resolution of the sensor. As described herein, a processor of a wireless implantable device may be configured to control or adjust a resolution setting of the sensor in order to configure the sensor to measure a physiological parameter at a first resolution or a second resolution. In some variations, a resolution setting may comprise one or more of a number of bits per sample, a voltage, a current, a sampling rate, a sampling duration, a number of samples, an over-sampling ratio (OSR), a frequency, a phase, an impedance, a filter cut-off frequency, combinations thereof, and the like.

f. Processor

Generally, a processor (e.g., CPU) described herein may receive, transmit and/or process data and/or other signals, and/or control one or more components of the system (e.g., control one or more circuit blocks of a wireless monitor). The processor may be configured to receive, process, compile, compute, store, access, read, write, transmit and/or generate data and/or other signals. Additionally, or alternatively, one or more blocks of the processor of a wireless monitor may be configured to control one or more other blocks of the processor and/or one or more components (e.g., transducer, power circuit, memory, sensor, and the like) of a wireless monitor. A processor, as described herein, may be included in one or more of a wireless monitor, an external wireless device, and the like.

In some variations, a processor (140) of a wireless implantable device (100) may be configured to process a parameter (e.g., a physiological parameter of a patient) measured by a sensor, and generate parameter data (e.g., physiological parameter data). In some variations, the processor (140) may be configured to compare a measured physiological parameter (e.g., at a first resolution) to a predetermined threshold (e.g., a first threshold) in order to generate physiological parameter data. For example, physiological parameter data may signify if the measured physiological parameter value is above or below a predetermined threshold value. In some variations, physiological parameter data may comprise one or more of a mean, a median, a sum, a minimum, a maximum of a plurality of physiological parameter measurements (e.g., measured at a first resolution), combinations thereof, and the like. In some variations, physiological parameter data may comprise a result of comparing one or more of a mean, a median, a sum, a minimum, a maximum of a plurality of physiological parameter measurements (e.g., at a first resolution), combinations thereof, and the like, to a predetermined threshold (e.g., a second threshold).

In some variations, a processor (140) may be configured to control one or more circuit blocks (e.g., circuits) of the wireless monitor. For example, the processor (140) may be configured to adjust a resolution setting of the sensor, as described herein. As another example, the processor (140) may be configured to generate one or more signals (e.g., a trigger signal) to enable/disable one or more circuit blocks of the wireless monitor. In some variations, a processor (140) may comprise a timer circuit to generate a timer signal, wherein the processor (140) may generate the trigger signal based on the timer signal.

In some variations, a processor (140) of a wireless implantable device (100) may be configured to monitor one or more circuit blocks of the wireless implantable device (100). For example, the processor (140) may be configured to monitor one or more energy storage devices (130) of the wireless implantable device (100). In some variations, a processor (140) may comprise an energy storage monitoring circuit, which may comprise one or more of a battery monitoring circuit, a capacitor monitor circuit, combinations thereof, and the like. For example, the processor (140) or an energy storage monitoring circuit may be configured to measure one or more energy storage device parameters. In some variations, a processor (140) may be configured to generate one or more mode selection signals for a load circuit (150) based on the measured energy storage device parameter.

In some variations, a processor (140) of a wireless implantable device (100) may be configured to digitize an analog signal (e.g., a signal measured by a sensor or a sensing transducer). In some variations, a processor (140) may comprise a data communication circuit that may be a data receiver, which may be configured to access or receive data and/or other signals from one or more of a transducer, a sensor (e.g., pressure sensor) and a storage medium (e.g., memory, flash drive, memory card). For example, the processor may comprise one or more of a signal receiver (e.g., detecting an interrogation signal), an envelope detector circuit, an amplifier (e.g., a low-noise amplifier or LNA), a filter, a frequency detector circuit, a phase detector circuit, comparator circuits, decoder circuits, combinations thereof, and the like, to receive data and/or signals through the transducer.

In some variations, a processor (140) may comprise any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may comprise, for example, a general purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, a processor (140) of a wireless implantable device (100) may comprise one or more of an envelope detection circuit, an energy detector circuit, a power detector circuit, a voltage sensor, a time-to-digital converter (TDC) circuit, an integrator circuit, a sampling circuit, an analog-to-digital converter (ADC) circuit, a timer circuit, a clock, a counter, an oscillator, a phase-locked loop (PLL), a frequency locked loop (FLL), combinations thereof, and the like. In some variations, a processor (140) may comprise an amplifier, a phase detector, a frequency detector, a digital signal processor, an integrator, an adder circuit, a multiplier circuit, a finite state machine, combinations thereof, and the like, for performing computations.

In some variations, a processor (140) of a wireless implantable device (100) may comprise a data communication circuit that may be a data transmitter or a wireless transmitter, which may be configured to generate or transmit data and/or other signals through one or more of a transducer, a storage medium, and the like. For example, a processor (140) of a wireless implantable device (100) may comprise one or more of a signal transmitter, an uplink data transmitter, an oscillator, a power amplifier, a mixer, an impedance matching circuit, a switch, a driver circuit, combinations thereof, and the like, to generate or transmit data and/or signals via the transducer.

In some variations, a first processor may be included in a wireless monitor and a second processor may be included in an external wireless device.

g. Memory

Generally, an implantable device, a wireless monitor and/or the wireless device described herein may comprise a memory configured to store data and/or information. In some variations, the memory may be of one or more types including, but not limited to, random access memory (RAM), static RAM (SRAM), dynamic RAM (DRAM), resistive random-access memory (ReRAM or RRAM), magnetoresistive random-access memory (MRAM), ferroelectric random-access memory (FRAM), standard-cell based memory (SCM), shift registers, read-only memory (ROM), programmable read-only memory (PROM), erasable programmable read-only memory (EPROM), electrically erasable programmable read-only memory (EEPROM), flash memory (e.g., NOR, NAND), embedded flash, volatile memory, non-volatile memory, one time programmable (OTP) memory, combinations thereof, and the like.

In some variations, the memory may store instructions and/or data to cause the processor to execute modules, processes, and/or functions (e.g., executing a search algorithm) associated with a wireless monitor and/or an external wireless device. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) may be non-transitory in the sense that it may not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In some variations, the memory may be configured to store sensor data (e.g., physiological parameter data), received data and/or data generated by the wireless monitor (e.g., data generated by a processor) and/or the external wireless device. In some variations, the memory of a wireless monitor may be configured to store data generated upon processing signals sensed by a sensor (e.g., blood pressure data sensed by a pressure sensor that may be included in a wireless monitor). In some variations, the memory may be configured to store data temporarily or permanently.

h. Wireless Transmitter

Generally, a wireless transmitter of a wireless implantable device or a wireless monitor may be configured to wirelessly transmit one or more wireless signals, data, command and/or power. For example, a wireless transmitter of a wireless implantable device (100) may comprise one or more of a signal transmitter, an uplink data transmitter, an oscillator, a power amplifier, a mixer, an impedance matching circuit, a switch, a driver circuit, combinations thereof, and the like, to generate and/or wirelessly transmit data and/or signals via a transducer (110) of the wireless implantable device (100).

i. Wakeup Receiver Circuit

Generally, a wakeup receiver circuit of a wireless implantable device (100) may be configured to detect one or more wireless signals, data and/or commands received by a transducer (110) of the wireless implantable device (100), and generate one or more wakeup signals in response to the detected one or more wireless signals, data and/or commands. Such a wakeup signal may be configured to activate or turn on one or more circuit blocks of the wireless implantable device (100). For example, a wakeup receiver circuit may be configured to detect a wireless data uplink command transmitted by an external wireless device to a wireless implantable device (100), in order to configure the wireless implantable device for transmitting uplink data (e.g., sensor data) to the external wireless device. In some variations, a wakeup receiver circuit may comprise one or more of an envelope detector circuit, an amplifier, a filter, a mixer, a phase detector, a frequency detector, an energy detector, a comparator, an analog-to-digital converter, digital logic (e.g., to decode a wireless code or command sent to a wireless implantable device), combinations thereof, and the like.

j. Multiplexer Circuit

Generally, a multiplexer or multiplexer circuit described herein may be configured to decouple one or more of power signal, data signal and/or other signals in a wireless monitor. This may be done in order to avoid interference between these signals and ensure proper functioning of the wireless monitor. For example, a multiplexer in a wireless monitor may be configured to decouple a power signal from a data signal received by a transducer of the wireless monitor from an external wireless device such that the power signal is provided to the power circuit for power recovery and conditioning, and the data signal is provided to the processor for data recovery.

In some variations, the multiplexer may comprise one or more of transmit/receive switches, passive devices (e.g., diodes, relays, MEMS circuits, blockers, passive switches), circulators, frequency selection (e.g., using filters, impedance matching networks), direct wired connections, combinations thereof, and the like.

In some variations, the transmit/receive switches may be driven based on timing control or time multiplexing such that one or more of power signal, data signal and other signals are received by a wireless monitor at different times. In some variations, the transmit/receive switches may be driven based on amplitude selection wherein one or more of power signal, data signal and other signals have different amplitudes. In some variations, the transmit/receive switches may be driven based on frequency selection or frequency multiplexing wherein one or more of power signal, data signal and other signals have different frequencies. In some variations, the transmit/receive switches may be implemented using depletion-mode transistors to operate when the wireless monitor may not have power, stored energy or an established voltage rail.

D. Wireless Device

Generally, a wireless device or external wireless device may refer to any device that is physically separate from a wireless implantable device or a wireless monitor. In some variations, the external wireless device may comprise one or more blocks described herein in the context of the wireless implantable device including, but not limited to, a transducer, a power circuit, an energy storage device, a load circuit, a sensor, a processor, a memory, a wireless transmitter, a wakeup receiver circuit, a multiplexer circuit, combinations thereof, and the like. Variations of these blocks as explained herein in the context of a wireless implantable device are applicable here as well.

In some variations, the transducer of the external wireless device may comprise a plurality of ultrasonic transducer elements or an ultrasonic array configured to exchange wireless signals (transmit and/or receive) with one or more wireless implantable devices. As another example, in some variations, the transducer of the external wireless device may comprise one or more RF coils and/or RF antennas. In some variations, the processor of the external wireless device may perform one or more of processing data and/or signals received from one or more wireless monitors, processing data received from one or more other wireless devices, combinations thereof, and the like.

In some variations, an external wireless device may perform one or more functions including, but not limited to, transmitting one or more of wireless power, data and other signals to one or more wireless implantable devices, receiving one or more of wireless data and other signals from one or more wireless implantable devices, processing data and/or signals, performing sensing and/or actuation (e.g., measuring blood pressure, heart rate, heart rate variability, ECG, EKG, thoracic impedance, breathing rate or respiration, patient activity levels, heart sounds, temperature, body weight, blood glucose, blood oxygen, combinations thereof, and the like), storing data or information in memory, communicating with other external wireless devices (e.g., tablet, phone, computer) via wires and/or using wireless links (e.g., Bluetooth), displaying or providing data or information (e.g., visual display on a screen or a monitor, audio signals), generating alerts/notifications (e.g., visual, audio, vibration) to a user (e.g., patient, nurse, doctor), combinations thereof, and the like.

In some variations, an external wireless device may be located at one or more locations including, but not limited to, outside the body (e.g., as a wearable device, a strap, a belt, a handheld device, a probe connected to a measurement setup, a device placed on skin, a device attached to skin using an adhesive, a device attached to skin using other techniques, a device not touching the patient, a laptop, a computer, a mobile phone, a smartwatch, and the like), permanently implanted inside the body (e.g., implanted under the skin, along the outer wall of an organ, under a muscle, outside the heart wall, and the like), temporarily implanted (e.g., for a predetermined amount of time) inside the body (e.g., located on a catheter or a probe inserted through a blood vessel, esophagus or the chest wall, used during surgery or procedure), combinations thereof, and the like. In some variations, the external wireless device may have different shapes or forms, including but not limited to, planar, conformal to the body or an organ, flexible, stretchable, flat, shaped like a probe, and the like.

In some variations, the external wireless device may further comprise a communication device configured to permit a user and/or health care professional to control one or more of the devices of the wireless system. The communication device may comprise a network interface configured to connect the external wireless device to another system (e.g., Internet, remote server, database) by wired or wireless connection. In some variations, the external wireless device may be in communication with other devices (e.g., cell phone, tablet, computer, smartwatch, and the like) via one or more wired and/or wireless networks. In some variations, the network interface may comprise one or more of a radiofrequency receiver/transmitter, an optical (e.g., infrared) receiver/transmitter, an acoustic or ultrasonic receiver/transmitter, and the like, configured to communicate with one or more devices and/or networks. The network interface may communicate by wires and/or wirelessly with one or more of the external wireless device, network, database, and server.

The network interface may comprise RF circuitry configured to receive and/or transmit RF signals. The RF circuitry may convert electrical signals to/from electromagnetic signals and communicate with communication networks and other communication devices via the electromagnetic signals. The RF circuitry may comprise well-known circuitry for performing these functions, including but not limited to, an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

The communication device may further comprise a user interface configured to permit a user (e.g., subject or patient, predetermined contact such as a partner, family member, health care professional, etc.) to control the external wireless device. The communication device may permit a user to interact with and/or control an external wireless device directly and/or remotely. For example, a user interface of the external wireless device may include an input device for a user to input commands and an output device for a user to receive output (e.g., blood pressure readings on a display device).

In some variations, an output device of the user interface may output one or more of information about the coupling of an external wireless device to tissue or skin, information about the wireless link between the external wireless device and the wireless monitor (e.g., has a reliable link been established), data (e.g., physiological parameter data) measured by one or more of the wireless monitor and the external wireless device, combinations thereof, and the like. In some variations, an output device of the user interface may comprise one or more of a display device and audio device. Data analysis generated by a server may be displayed by the output device (e.g., display) of the external wireless device. Data used in finding an optimal transducer configuration or ensuring that an external wireless device is optimally coupled to tissue may be received through the network interface and output visually and/or audibly through one or more output devices of the external wireless device. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, an audio device may audibly output one or more of any data, commands, instructions to a user, alarms, notifications, and the like. For example, the audio device may output an audible alarm when the link between a wireless monitor and an external wireless device is disturbed or broken and manual adjustment by a user may be needed. In some variations, an audio device may comprise at least one of a speaker, piezoelectric audio device, magnetostrictive speaker, and/or digital speaker. In some variations, a user may communicate with other users using the audio device and a communication channel. For example, a user may form an audio communication channel (e.g., VoIP call) with a remote health care professional.

In some variations, the user interface may comprise an input device (e.g., touch screen) and output device (e.g., display device) and be configured to receive input data from one or more of the wireless monitor, an external wireless device, network, database, and server. For example, user control of an input device (e.g., keyboard, buttons, touch screen) may be received by the user interface and may then be processed by processor and memory for the user interface to output a control signal to the wireless monitor. Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the external wireless device.

### II. Methods

Described herein are methods for energy-efficient and reliable operation of wireless implantable devices and/or for monitoring a patient, using any of the systems and devices described herein. Generally, a wireless system or device may implement one or more of the methods described herein, or any sub-set of the one or more methods described herein, or a combination of methods or sub-sets thereof. One or more methods described here, or steps therein, may be applied to a plurality of wireless implantable devices and/or wireless monitors.

In some variations, energy-efficient monitoring using a wireless monitor may be facilitated by using a plurality of sensing resolutions. In some variations, a method of monitoring a patient may comprise one or more of the following steps, including but not limited to, measuring a physiological parameter of the patient at a first resolution using a wireless monitor, generating physiological parameter data based on the measured physiological parameter at the first resolution, measuring the physiological parameter of the patient at a second resolution using the wireless monitor based at least in part on the physiological parameter data, and estimating a physiological state of the patient based at least in part on the measured physiological parameter at the second resolution.

In some variations, wireless charging of an energy storage device may utilize a battery and capacitor. In some variations, a method of wirelessly charging a battery may comprise one or more of the following steps, including but not limited to, receiving wireless power using a transducer, recovering at least a portion of the received wireless power using a first power circuit, storing at least a portion of the recovered wireless power from the first power circuit in a capacitor as capacitor energy, and charging the battery using at least a portion of the capacitor energy during an absence of receiving the wireless power.

In some variations, a wireless implantable device may be operated in a plurality of modes. In some variations, a method of operating a wireless implantable device may comprise one or more of the following steps, including but not limited to, measuring an energy storage device parameter of an energy storage device of the wireless implantable device, generating a mode selection signal based on the measured energy storage device parameter, and configuring a load circuit to perform a predetermined function in a first mode or a second mode based on the mode selection signal. In some variations, the method may further comprise one or more of the following steps, including but not limited to, receiving wireless power using a transducer of the wireless implantable device, recovering at least a portion of the received wireless power using a power circuit of the wireless implantable device, and generating one or more supply voltages for the load circuit using a power detector circuit of the wireless implantable device.

In some variations, a wireless implantable device may be duty-cycled to reduce energy consumption. In some variations, a method of operating an implantable device may comprise one or more of the following steps, including but not limited to, generating a trigger signal for a wakeup receiver circuit based on a timer signal using a processor, operating the wakeup receiver circuit only upon receiving the trigger signal, receiving a wireless signal using a transducer, and generating a wakeup signal using the wakeup receiver circuit in response to the wireless signal. In some variations, a method of operating an implantable device may comprise one or more of the following steps, including but not limited to, monitoring one or more energy storage device parameters using an energy storage monitoring circuit, generating a trigger signal for the energy storage monitoring circuit based on the monitoring of the one or more energy storage device parameters, and operate the energy storage monitoring circuit only upon receiving the trigger signal.

In some variations, a patient's cardiovascular pressure may be monitored. In some variations, a method of cardiovascular pressure monitoring may comprise one or more of the following steps, including but not limited to, measuring cardiovascular pressure using a wireless monitor implanted in a patient, measuring at least one other physiological parameter of the patient, and determining a patient status based at least in part on the cardiovascular pressure and the at least one other physiological parameter of the patient.

Also described herein are methods of powering an implantable device in the body using energy harvesting. Methods of exchanging wireless signals and measuring pressure using a single transducer (e.g., an ultrasonic transducer) of a wireless monitor are also described herein.

A. Energy-Efficient Monitoring Using Plurality of Sensing Resolutions

For effective treatment and disease management, frequent or continuous monitoring of a physiological parameter of a patient (e.g., several times per day) may be desirable, as opposed to infrequent monitoring (e.g., once per day). For example, monitoring intracardiac pressure of a patient with cardiovascular diseases frequently throughout the day may be useful for detecting any adverse cardiac events, monitoring changes in cardiac parameters with exercise, walking, posture, diet, and the like, thereby potentially gaining a better understanding of the patient's disease state. However, measuring a physiological parameter with clinically required resolutions frequently throughout the day may be energy intensive. This may be challenging when monitoring a physiological parameter is performed using a miniature implantable device due to its limited energy budget.

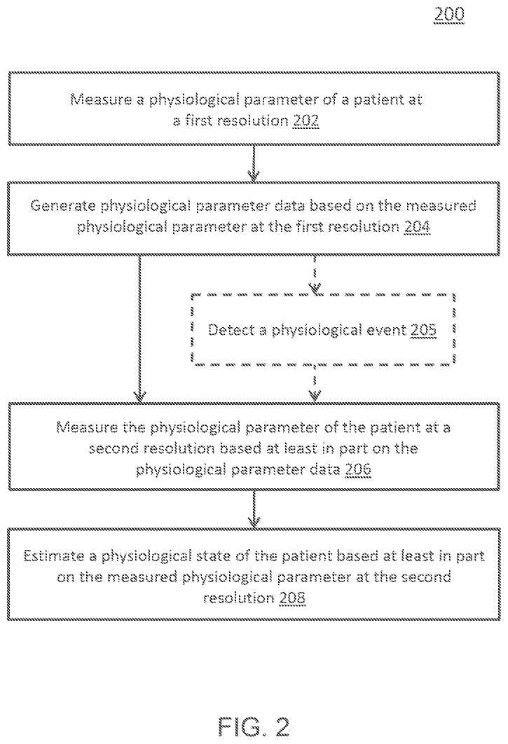

FIG. 2 is a flowchart that generally describes a method of monitoring a patient (200). The method (200) may comprise the steps of measuring a physiological parameter of a patient at a first resolution (202), generating physiological parameter data based on the measured physiological parameter at the first resolution (204), measuring the physiological parameter of the patient at a second resolution based at least in part on the physiological parameter data (206), and estimating a physiological state of the patient based at least in part on the measured physiological parameter at the second resolution (208). The first resolution, the second resolution, the physiological parameter of the patient, and the physiological parameter data, as described herein, are applicable to any of the methods described herein.

For example, intracardiac pressure of a patient may be measured at a first resolution (e.g., ±5 mmHg) and a mean of a plurality of such measurements may be computed. If the mean is determined to be above a predetermined threshold, the intracardiac pressure may be measured at a second resolution (e.g., ±1 mmHg). Measurement of intracardiac pressure at the second resolution may be used (e.g., by a doctor) to estimate a physiological state of the patient. In some variations, the second resolution may be a clinical-grade resolution.

In some variations, measuring the physiological parameter at the first resolution may consume lower energy compared to measuring the physiological parameter at the second resolution.

In some variations, for a given amount of energy consumption, measuring the physiological parameter at the first resolution (e.g., a coarse resolution) may allow more frequent monitoring of the patient compared to measuring the physiological parameter at the second resolution (e.g., a fine resolution). In some variations, measuring the physiological parameter at the first resolution may be used for screening (e.g., to detect a physiological event such as abnormally high intracardiac pressure), and measuring the physiological parameter at the second resolution may be used to collect higher-quality data for estimating a physiological state of the patient (e.g., a higher-resolution intracardiac pressure waveform which may be used for diagnosis or prognosis of the physiological event). In some variations, measuring the physiological parameter of the patient at the first resolution may be performed periodically at a predetermined repetition interval. In some variations, the method (200) of monitoring a patient may be performed periodically at a predetermined repetition interval.

Optionally, the method (200) of monitoring a patient may comprise the step of detecting a physiological event (205) based at least in part on one or more of the measured physiological parameter at the first resolution and the physiological parameter data. In some variations, the detected physiological event may comprise one or more of arrhythmia, atrial fibrillation, ventricular tachycardia, sleep apnea, abnormally high blood pressure, abnormally low blood pressure, abnormal pressure variation, abnormal heart rate, abnormal heart rate variability, combinations thereof, and the like.

In some variations, the method (200) of monitoring a patient may further comprise the step of storing one or more of the measured physiological parameter at the first resolution, the measured physiological parameter at the second resolution, the physiological parameter data, resolution data, combinations thereof, and the like, in a memory of the wireless monitor. In some variations, resolution data may comprise any information relating to the values of the first and/or the second resolution. For example, for the measurement of intracardiac pressure, if the first resolution is ±5 mmHg, and the second resolution is ±1 mmHg, resolution data may comprise one or more of the values ±5 mmHg, ±1 mmHg, a ratio of these values, and any other setting or parameter that may be used to derive these values. In some variations, the method (200) of monitoring a patient may further comprise the step of wirelessly transmitting one or more of the measured physiological parameter at the first resolution, the measured physiological parameter at the second resolution, the physiological parameter data, resolution data, combinations thereof, and the like, from the wireless monitor to a wireless device. In some variations, the wireless device may comprise an external wireless device configured to be disposed physically separate from the wireless monitor (e.g., external to the patient).

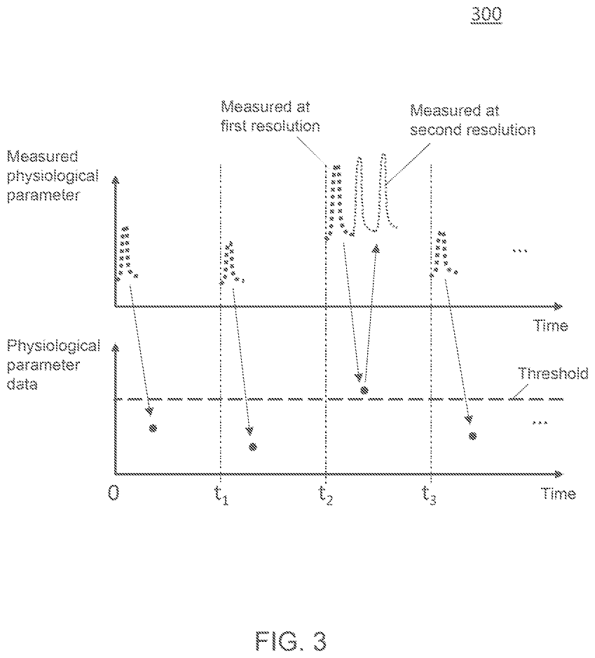

FIG. 3 shows a timing diagram of a method of monitoring a patient (300). As shown in FIG. 3, a physiological parameter of a patient may be measured periodically (at times 0, t₁, t₂, t₃, and so on) at a first resolution. The first resolution may comprise a coarse amplitude and/or timing resolution. For example, intracardiac pressure may be measured periodically at an amplitude resolution of ±5 mmHg and/or at a timing resolution of about 100 ms (i.e., at a frequency of about 10 Hz). Physiological parameter data (e.g., a maximum of the intracardiac pressure measurements at the first resolution) may be generated, as shown, and compared to a threshold. If the physiological parameter data is determined to be greater than the threshold, the physiological parameter may be measured at a second resolution. For example, the second resolution may comprise a fine amplitude resolution of ±1 mmHg and/or a fine timing resolution of about 10 ms (i.e., at a frequency of about 100 Hz).

In some variations, a second physiological parameter (e.g., heart rate, blood flow, patient activity, and the like) may be measured based at least in part on the physiological parameter data. For example, if measurement(s) of intracardiac pressure at a coarse resolution are determined to be above a threshold, measurement of a second physiological parameter such as blood flow may be triggered, alternatively or in addition to measurement of intracardiac pressure at a fine resolution (e.g., relative to the coarse resolution).

In some variations, the method (200) may be implemented by one or more of a wireless monitor, an external wireless device, or a combination thereof. In some variations, a wireless monitor may comprise a sensor configured to measure a physiological parameter of a patient at a first resolution, a processor configured to generate physiological parameter data based on the measured physiological parameter of the patient at the first resolution. The sensor may be configured to measure the physiological parameter of the patient at a second resolution based at least in part on the physiological parameter data. The wireless monitor, the sensor, the processor, the resolution, and the physiological parameter, as described herein, are applicable to any of the methods described herein. In some variations, the wireless monitor may be implanted in a patient. In some variations, an external wireless device may be configured to estimate a physiological state of the patient based at least in part on the measured physiological parameter at the second resolution. For example, the wireless monitor may be configured to wirelessly transmit one or more of the measured physiological parameter at the first resolution, the measured physiological parameter at the second resolution, the physiological parameter data, and resolution data to the external wireless device, prior to estimation of the physiological state of the patient.

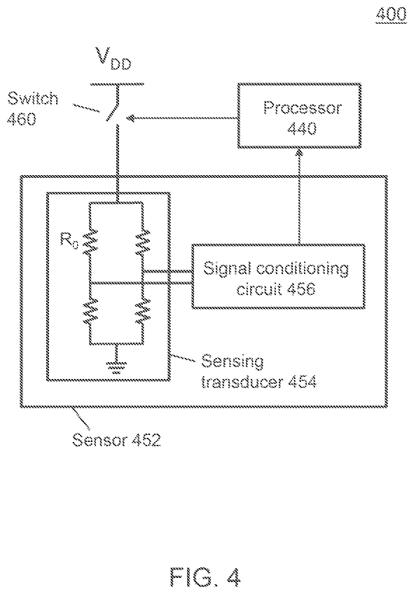

FIG. 4 depicts a wireless monitor (400) comprising a sensor (452) and a processor (440). The sensor (452) may comprise a sensing transducer (454) and a signal conditioning circuit (456). As an example, the sensing transducer (454) may comprise a piezoresistive pressure transducer configured to sense intracardiac pressure. As an example, the signal conditioning circuit (456) may comprise one or more of a front-end amplifier, an anti-aliasing filter, an analog-to-digital converter, a reference generator, a bias generator, a clock generator, combinations thereof, and the like. The piezoresistive pressure transducer may have a bridge resistance denoted by Ro in each branch. In some variations, the processor (440) may be configured to duty-cycle or power-gate the sensing transducer (454) or provide a supply voltage (V_{DD}) to the sensing transducer (454) by controlling a switch (460). When the switch is ON, the sensing transducer (454) dissipates power (P_{bridge}), based on the equation given by:

$${P}_{bridge} = \frac{{V}_{DD}^{2}}{{R}_{0}} \qquad ( 1 )$$

If the switch is ON for a sampling duration denoted by Tₛₐₘₚ, the total energy consumed by the sensing transducer (E_{bridge}) may be given by:

$${E}_{bridge} = \frac{{V}_{DD}^{2}}{{R}_{0}} \times {T}_{samp} \qquad ( 2 )$$

A large Tₛₐₘₚ (or settling time) may be required to achieve a fine sampling resolution (since a large sampling capacitance may be required to keep thermal noise below a certain limit). Thus, a fine sampling resolution (relative to a coarse resolution) may be related to relatively larger energy consumption.

In some variations, the sensor may comprise one or more resolution settings of the sensor. The resolution setting, as described herein, is applicable to any of the systems or methods described herein. For example, the resolution setting may comprise the sampling duration, Tₛₐₘₚ. In some variations, the processor (440) may be configured to control the sampling duration, Tₛₐₘₚ, by controlling the duration for which the switch (460) is ON. For example, the first resolution may correspond to a relatively small value of the sampling duration (low energy consumption) and the second resolution may correspond to a relatively large value of the sampling duration (greater energy consumption). In some variations, the resolution setting may comprise a supply voltage (V_{DD}) provided to the sensing transducer (454). For example, the processor (440) may be configured to adjust the value of the supply voltage (V_{DD}) provided to the sensing transducer (454), alternatively or in addition to adjusting sampling duration, in order to adjust the resolution of the sensor (452).

In some variations, the resolution setting may comprise a predetermined number of samples and/or a sampling rate. For example, a first resolution may comprise relatively fewer samples or a relatively lower sampling rate of a physiological parameter (leading to low energy consumption), and a second resolution may comprise a relatively larger number of samples or a relatively larger sampling rate of the physiological parameter (leading to greater energy consumption). In some variations, a plurality of measurements of the physiological parameter at the first and/or the second resolution may be averaged in order to improve the signal-to-noise ratio of the measurement.

In some variations, the processor (440) of the wireless monitor (400) may be configured to control the sensor to measure the physiological parameter of the patient at the first resolution periodically at a predetermined repetition interval. For example, the processor (440) may comprise a timer circuit to generate a periodic timer signal for triggering the sensor (452) to operate periodically. In some variations, one or more of the measured physiological parameter at the first resolution, the measured physiological parameter at the second resolution, and the physiological parameter data, may comprise digital bits. For example, the signal conditioning circuit (456) of the sensor (452) may comprise an analog-to-digital converter to generate digital bits representing the measured physiological parameter at the first and/or the second resolution.

In some variations, the wireless monitor (400) may comprise a memory configured to store one or more of the measured physiological parameter at the first resolution, the measured physiological parameter at the second resolution, the physiological parameter data, resolution data, the resolution setting, combinations thereof, and the like. The memory, as described herein, is applicable to any of the systems or methods described herein. In some variations, the wireless monitor (400) may comprise a wireless transmitter configured to wirelessly transmit one or more of the measured physiological parameter at the first resolution, the measured physiological parameter at the second resolution, the physiological parameter data, resolution data, the resolution setting, combinations thereof, and the like, to a wireless device. The wireless transmitter and the wireless device, as described herein, are applicable to any of the systems or methods described herein. In some variations, the wireless device may be an external wireless device configured to be disposed physically separate from the wireless monitor (e.g., external to the patient).

In some variations, the sensor of the wireless monitor may be configured to measure a physiological parameter of a patient at a first resolution by default, and measurement at a second resolution may be triggered only based upon physiological parameter data. In some variations, the sensor of the wireless monitor may be additionally configured to measure the physiological parameter of the patient at the second resolution irrespective of the physiological parameter data (e.g., periodically). In some variations, a sensor of a wireless monitor may be configured to measure a physiological parameter of a patient at a first resolution and a second resolution periodically, wherein measurements at the first resolution may be more frequent than measurements at the second resolution.

B. Wireless Charging of an Energy Storage Device

In some variations, a wireless implantable device may comprise an energy storage device, such as a rechargeable battery, that may need to be wirelessly recharged by an external wireless device. A rechargeable battery may have a maximum rate (e.g., a maximum current) at which it may be recharged. Such a limit on the charging rate of the energy storage device may mean that even if an external wireless device is able to transfer high power levels to the wireless implantable device, the energy storage device may still take a relatively long time to charge. Furthermore, in some wireless charging systems, if the wireless powering link is interrupted, the energy storage device may stop charging. As a result, a user (e.g., a patient) may be required to use or engage an external wireless device for a long duration to fully charge the wireless implantable device, limited by the charging rate of the energy storage device and to overcome any charging interruptions. This may be undesirable since users such as patients may not comply with a long charging routine, which may lead to an inadequate management of their disease. The methods described herein mitigate this challenge and enable patients to transfer the requisite amount of energy to the wireless implantable device in a shorter duration of time. While systems and methods described herein refer to the charging of a battery, they may also be applicable to the charging of other types of energy storage devices such as a capacitor, a super-capacitor, combinations thereof, and the like.

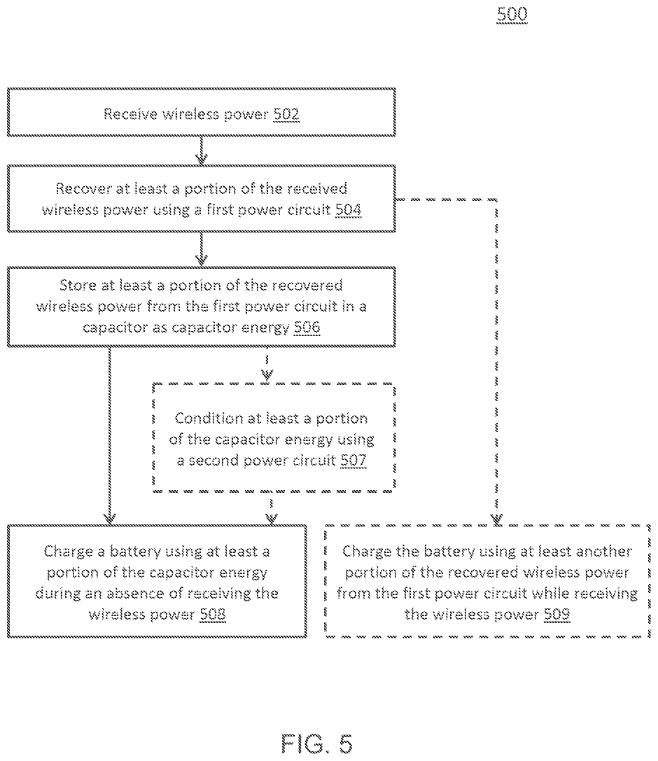

FIG. 5 is a flowchart that generally describes a method of wirelessly charging an energy storage device (e.g., a battery) (500). The method (500) may comprise the steps of receiving wireless power (502), recovering at least a portion of the received wireless power using a first power circuit (504), storing at least a portion of the recovered wireless power from the first power circuit in a capacitor as capacitor energy (506), and charging an energy storage device (e.g., a battery) using at least a portion of the capacitor energy during an absence of receiving the wireless power (508). In some variations, the method (500) may further comprise charging the energy storage device (e.g., battery) using at least another portion of the recovered wireless power from the first power circuit while receiving the wireless power (509). The wireless power, the power circuit, and the energy storage device, as described herein, are applicable to any of the methods described herein.

Optionally, the method (500) may comprise conditioning at least the portion of the capacitor energy using a second power circuit (507) prior to charging the battery during the absence of receiving the wireless power. In some variations, the method (500) may further comprise conditioning at least another portion of the recovered wireless power from the first power circuit using a second power circuit prior to charging the battery while receiving the wireless power. In some variations, the method (500) may comprise recovering at least another portion of the received wireless power from the transducer using a second power circuit and charging the battery using at least a portion of the recovered wireless power from the second power circuit while receiving the wireless power. In some variations, the method (500) may comprise conditioning at least the portion of the recovered wireless power from the first power circuit using a third power circuit prior to storing at least the portion of the recovered wireless power from the first power circuit in the capacitor as capacitor energy.

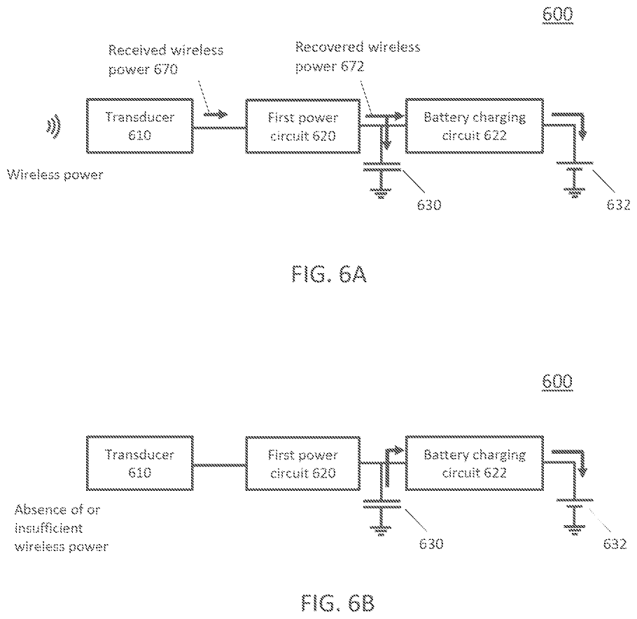

FIGS. 6-10 illustrate variations of the wireless charging methods described herein. FIG. 6A depicts a wireless battery charging system (600) comprising a first power circuit while receiving wireless power. FIG. 6B depicts the wireless battery charging system (600) during an absence of receiving the wireless power (or receiving insufficient wireless power). The wireless battery charging system (600) may comprise a transducer (610) configured to receive wireless power, a first power circuit (620) coupled to the transducer and configured to recover at least a portion of the received wireless power (670) by the transducer (610), a capacitor (630) coupled to the first power circuit (620) and configured to store at least a portion of the recovered wireless power (672) from the first power circuit (620) as capacitor energy, and a battery (632) coupled to the capacitor (630) and configured to charge using at least a portion of the capacitor energy during an absence of receiving the wireless power. Arrows in this figure (and the following figures of this sub-section) conceptually illustrate a flow of majority of the power in the wireless battery charging system. In some variations, the wireless battery charging system may be a wireless implantable device implanted in a patient. In some variations, the first power circuit may be configured to recover AC voltage generated at the terminals of the transducer (610) to generate one or more DC voltage rails. In some variations, the first power circuit may comprise one or more of an AC-DC converter, a re-configurable AC-DC converter, a rectifier, a re-configurable rectifier, a DC-DC converter, a re-configurable DC-DC converter, a linear regulator, a switching regulator, a switched-capacitor voltage regulator, a voltage limiter, combinations thereof, and the like.

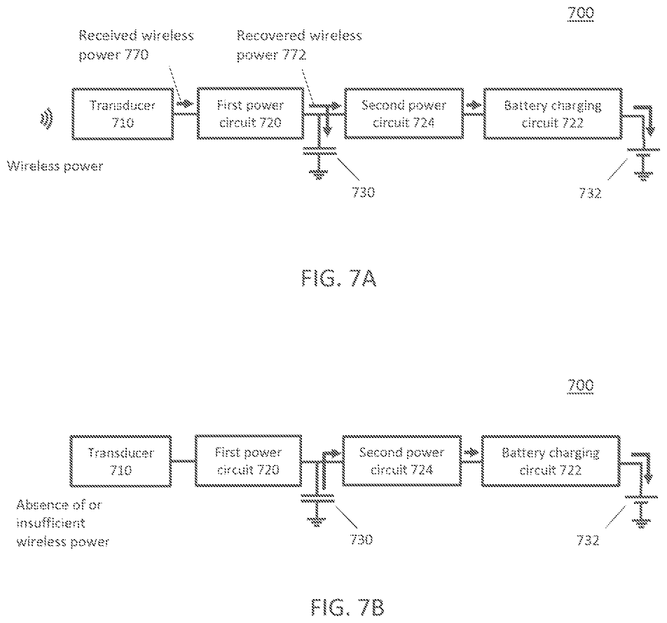

FIG. 7A depicts a wireless battery charging system (700) comprising a first power circuit (720) and a second power circuit (724) while receiving wireless power. FIG. 7B depicts the wireless battery charging system (700) during an absence of receiving the wireless power (or receiving insufficient wireless power). In some variations, in addition to a transducer (710), a first power circuit (720), a capacitor (730) and a battery (732), a wireless battery charging system (700) may comprise a second power circuit (724) coupled to the capacitor (730) and configured to condition at least the portion of the capacitor energy prior to charging the battery (732) during the absence of receiving the wireless power, as shown in FIG. 7B. In some variations, the wireless battery charging system (700) may comprise a second power circuit (724) coupled to the first power circuit (720) and configured to condition at least the another portion of the recovered wireless power (772) from the first power circuit (720) prior to charging the battery (732) while receiving the wireless power.

In some variations, the first power circuit (720) may be configured to recover AC voltage generated at the terminals of the transducer (710) to generate one or more DC voltage rails. In some variations, the first power circuit (720) may comprise one or more of an AC-DC converter, a re-configurable AC-DC converter, a rectifier, a re-configurable rectifier, a DC-DC converter, a re-configurable DC-DC converter, a linear regulator, a switching regulator, a switched-capacitor voltage regulator, a voltage limiter, combinations thereof, and the like. In some variations, the second power circuit (724) may be configured to condition the power in order to accomplish one or more of transforming one DC voltage into another (e.g., step-up or step-down DC-DC conversion), transforming an impedance of the power circuit (e.g., to efficiently drive the battery charging circuit), combinations thereof, and the like. In some variations, the second power circuit (724) may comprise one or more of a DC-DC converter, a re-configurable DC-DC converter, a linear regulator, a switching regulator, a switched-capacitor voltage regulator, a voltage limiter, combinations thereof, and the like. In some variations, one or more of the first power circuit (720) and the second power circuit (724) may comprise wires directly connecting the input of the power circuit to its output.

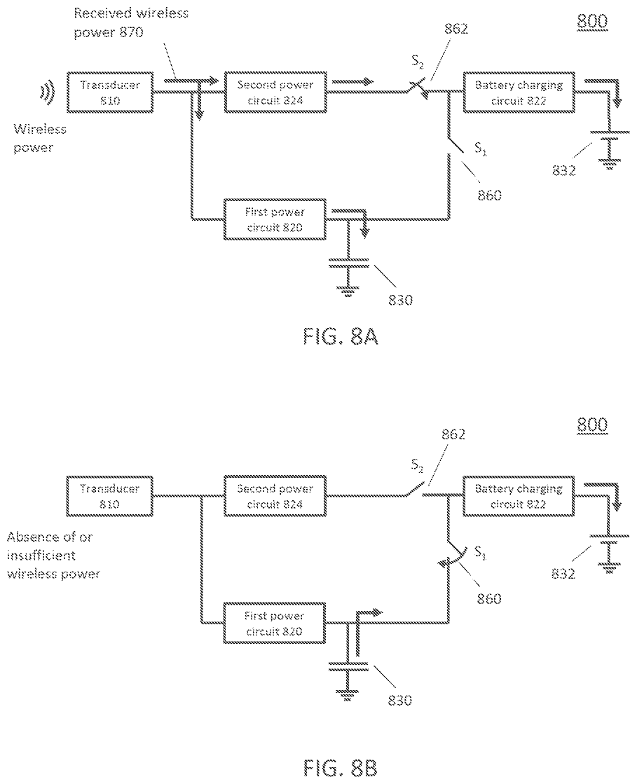

FIG. 8A depicts a wireless battery charging system (800) comprising a first power circuit (820) and a second power circuit (824) while receiving wireless power. FIG. 8B depicts the wireless battery charging system (800) during an absence of receiving the wireless power (or receiving insufficient wireless power). In some variations, in addition to a transducer (810), a first power circuit (820), a capacitor (830) and a battery (832), a wireless battery charging system (800) may comprise a second power circuit (824) coupled to the transducer (810) and configured to recover at least another portion of the received wireless power (870) from the transducer (810), wherein the battery (832) may be configured to charge using at least a portion of the recovered wireless power from the second power circuit (824) while receiving the wireless power.

In some variations, the wireless battery charging system (800) may comprise at least a first switch (860), labeled as S₁ in FIG. 8B, coupled between the capacitor (830) and the battery (832), wherein the at least first switch (860) may be configured to be on during an absence of receiving the wireless power. In some variations, the wireless battery charging system (800) may comprise at least a second switch (862), labeled as S₂ in FIG. 8A, coupled between the second power circuit (824) and the battery (832), wherein the at least second switch (862) may be configured to be on while receiving the wireless power. In some variations, the status of wireless power (presence, absence or whether received wireless power level is above or below a threshold) may be detected using a power detector circuit (e.g., an envelope detector circuit) configured to monitor one or more of a voltage, a current, an impedance, a power level, and an energy level of one or more of the transducer (810), the first power circuit (820), and the second power circuit (824). In some variations, the wireless battery charging system (800) may further comprise a processor or a controller to control the turning on and off of the switches S₁ and S₂. In some variations, the state of the switches S₁ and/or S₂ may be changed (on to off or vice versa) after a delay or a wait time upon detection of the presence or absence of the received wireless power. In some variations, the capacitor (830) may be disconnected from the battery charging circuit (822), e.g., by turning off switch S₁, when the voltage of the capacitor (830) falls below a threshold (e.g., below a predetermined threshold, below the battery voltage, below a threshold voltage of a DC-DC converter included in the battery charging circuit, and the like).

The battery (832) in FIGS. 8A and 8B may be charged via two independent or parallel paths during an absence or presence of wireless power. Such a circuit configuration may enable independent control over impedance or power matching for charging both the battery (832) and the capacitor (830) efficiently. For example, the design of the first power circuit (820) may be optimized to efficiently charge the capacitor (830), and the design of the second power circuit (824) may be independently optimized to efficiently charge the battery (832) while receiving the wireless power. Further, such a circuit architecture may offer an added degree of freedom to target different DC voltages at the output of the first power circuit (820) and at the output of the second power circuit (824).

In some variations, the first power circuit (820) and the second power circuit (824) may be configured to recover AC voltage generated at the terminals of the transducer (810) to generate one or more DC voltage rails. In some variations, the first power circuit (820) and the second power circuit (824) may comprise one or more of an AC-DC converter, a re-configurable AC-DC converter, a rectifier, a re-configurable rectifier, a DC-DC converter, a re-configurable DC-DC converter, a linear regulator, a switching regulator, a switched-capacitor voltage regulator, a voltage limiter, combinations thereof, and the like. In some variations, one or more of the first power circuit (820) and the second power circuit (824) may comprise wires directly connecting the input of the power circuit to its output.

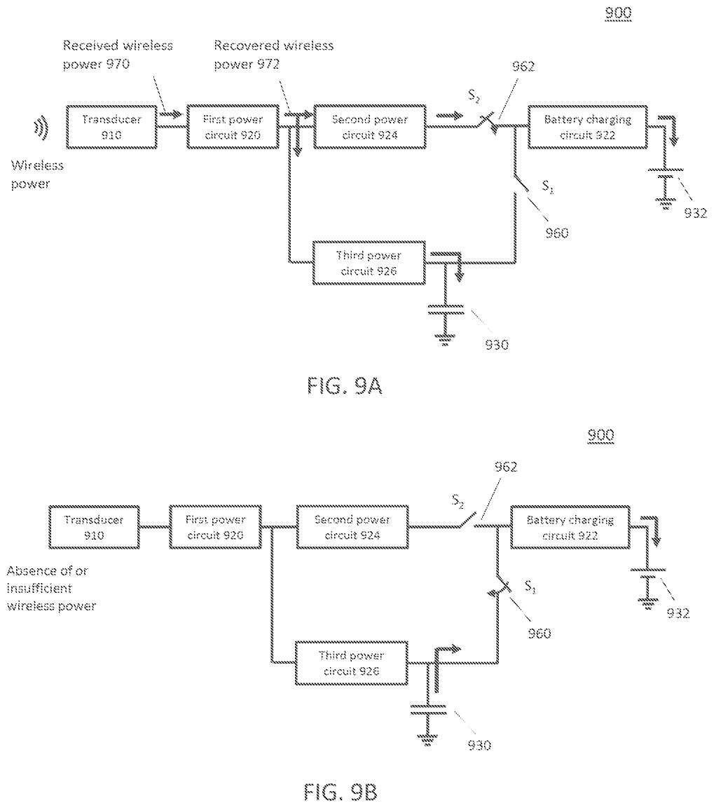

FIG. 9A depicts a wireless battery charging system (900) comprising a first power circuit (920), a second power circuit (924) and a third power circuit (926), while receiving wireless power. FIG. 9B depicts the wireless battery charging system (900) during an absence of receiving the wireless power (or receiving insufficient wireless power). In some variations, in addition to a transducer (910), a first power circuit (920), a second power circuit (924), a capacitor (930) and a battery (932), a wireless battery charging system (900) may comprise a third power circuit (926) coupled to the first power circuit (920) and configured to condition at least the portion of the recovered wireless power (972) from the first power circuit (920) prior to storing at least the portion of the recovered wireless power (972) from the first power circuit (920) in the capacitor (930) as capacitor energy.

In some variations, the wireless battery charging system (900) may comprise at least a first switch (960), labeled as S₁ in FIG. 9B, coupled between the capacitor (930) and the battery (932), wherein the at least first switch (960) may be configured to be on during an absence of receiving the wireless power. In some variations, the wireless battery charging system (900) may comprise at least a second switch (962), labeled as S₂ in FIG. 9A, coupled between the second power circuit (924) and the battery (932), wherein the at least second switch (962) may be configured to be on while receiving the wireless power. In some variations, the status of wireless power (presence, absence or whether received wireless power level is above or below a threshold) may be detected using a power detector circuit (e.g., an envelope detector circuit) configured to monitor one or more of a voltage, a current, an impedance, a power level, and an energy level of one or more of the transducer (910), the first power circuit (920), the second power circuit (924), and the third power circuit (926). Advantages of the circuit architecture depicted in FIGS. 9A and 9B may be similar to the advantages of the circuit architecture depicted in FIGS. 8A and 8B discussed above.

In some variations, the first power circuit (920) may be configured to recover AC voltage generated at the terminals of the transducer (910) to generate one or more DC voltage rails. In some variations, the first power circuit (920) may comprise one or more of an AC-DC converter, a re-configurable AC-DC converter, a rectifier, a re-configurable rectifier, a DC-DC converter, a re-configurable DC-DC converter, a linear regulator, a switching regulator, a switched-capacitor voltage regulator, a voltage limiter, combinations thereof, and the like. In some variations, the second power circuit (924) and the third power circuit (926) may be configured to condition the power in order to accomplish one or more of transforming one DC voltage into another (e.g., step-up or step-down DC-DC conversion), transforming an impedance of the power circuit (e.g., to efficiently drive the battery charging circuit), combinations thereof, and the like. In some variations, the second power circuit (924) and the third power circuit (926) may comprise one or more of a DC-DC converter, a re-configurable DC-DC converter, a linear regulator, a switching regulator, a switched-capacitor voltage regulator, a voltage limiter, combinations thereof, and the like. In some variations, one or more of the first power circuit (920), the second power circuit (924), and the third power circuit (926), may comprise wires directly connecting the input of the power circuit to its output.

While different exemplary illustrations of a wireless battery charging system are described above, in some variations, one or more of the power circuits may not be provided, or wires may take the place of one or more power circuits. For example, wires may take the place of the first power circuit in FIGS. 9A and 9B, resulting in a wireless battery charging system similar to that depicted in FIGS. 8A and 8B.

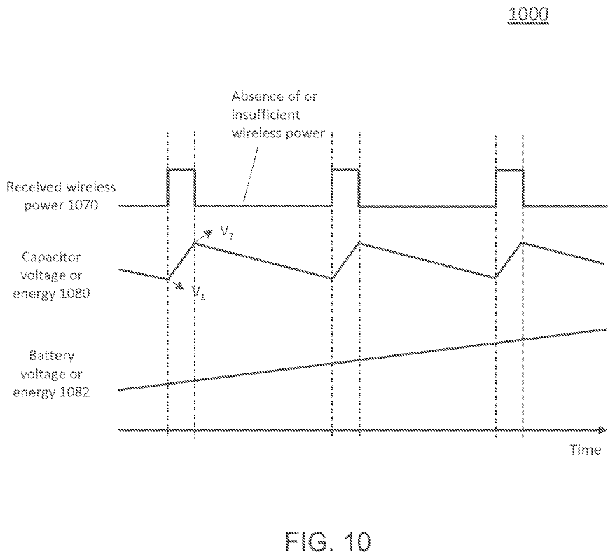

In some variations, the battery may have a capacity of less than about 100 milli-Watthour. In some variations, the capacitor may have a capacitance between about 0.1 nF and about 100 μF. FIG. 10 shows a conceptual timing diagram of a variation of a method of wirelessly charging a battery. During a presence of received wireless power (1070), a capacitor of a wireless battery charging system may be charged, resulting in an increase in the capacitor voltage or energy (1080), as shown in FIG. 10. For example, the capacitor may be charged from a voltage of V₁ to a voltage of V₂. During an absence of wireless power, or when insufficient wireless power is received, the battery may be charged using capacitor energy. This is illustrated in FIG. 10 by an increase in the battery voltage or energy (1082) during an absence of wireless power, and a corresponding decrease in the capacitor voltage or energy (1080).

An example implementation of the wireless battery charging system is presented herein. Consider a battery of a wireless implantable device with a maximum charging rate of 500 μW. Assume that the system requires charging the battery at this rate for 10 ms, i.e., delivering 5 μJ energy to the battery. However, if wireless power remains on only for 2 ms, and is off for the remaining 8 ms (e.g., due to an interruption of wireless power, or movement of the wireless implantable device), the battery would receive only a fifth of the required energy. Using the wireless battery charging system described herein, assume that the capacitor discharges from voltage V₂ (e.g., 6 V) to voltage V₁ (e.g., 4 V) while charging the battery at power, P_{charge}, of 500 μW for duration, T_{charge}, of 8 ms when the wireless power is absent (e.g., very low or no wireless power is received by the wireless implantable device). Thus, based on these example values, and ignoring any energy loss while charging the battery for simplicity, the required capacitance (C) can be computed to be about 400 nF using:

½C(V₂²−V₁²)=P_{charge}×T_{charge} (3)

In some variations, the capacitor may comprise a first rate of storing wireless power recovered by a power circuit, and the battery may comprise a second rate of charging using the capacitor energy. In some variations, the first rate may be greater than the second rate. For example, this may be advantageous in scenarios where a battery of a wireless implantable device may have a limited charging rate, thereby allowing a user to quickly store requisite energy on a capacitor of the wireless implantable device (due to its faster charging rate), wherein the battery may continue to charge using the capacitor energy even after the user may have disengaged or interrupted wireless power.

In some variations, any wireless battery charging system described above may comprise a battery charging circuit coupled to the battery and configured to charge the battery. In some variations, the battery charging circuit may comprise one or more of a constant-voltage (CV) charging circuit, a constant-current (CC) charging circuit, a trickle charging circuit, a pulsed charging circuit, a DC-DC converter, a re-configurable DC-DC converter, a linear regulator, a switching regulator, a switched-capacitor voltage regulator, a voltage limiter configured to limit the voltage of the battery during charging, combinations thereof, and the like. In some variations of the systems illustrated in FIGS. 8A, 8B, 9A, and 9B, the battery charging circuit may comprise a plurality of DC-DC converters or a re-configurable DC-DC converter, wherein a configuration or a gain of the DC-DC converter may be selected based on whether switch S₁ or S₂ is on. For example, such reconfigurability may be advantageous for targeting a certain voltage (e.g., 3-4 V) at the battery during charging of the battery, irrespective of the voltages at the output of the second power circuit and the voltage on the capacitor.

The transducer, as described herein, is applicable to any of the systems or methods described herein. In some variations, the transducer may comprise an acoustic transducer, and wireless power may comprise acoustic power. In some variations, the acoustic transducer may comprise an ultrasonic transducer, and the acoustic power may comprise ultrasonic power. In some variations, the transducer may comprise a plurality of transducers or a plurality of transducer elements. For example, in the variation of FIGS. 8A and 8B, the transducer (810) may comprise a first transducer coupled to the first power circuit (820), and a second transducer coupled to the second power circuit (824), wherein the first power circuit (820) may be configured to recover the wireless power received by the first transducer, and the second power circuit (824) may be configured to recover the wireless power received by the second transducer.

C. Operating a Wireless Implantable Device in a Plurality of Modes

In some applications, a wireless implantable device may be configured to operate in a first mode, wherein it may perform a task or a function (e.g., sensing, stimulation, and the like) without requiring wireless power or a wireless command from a user each time the task or the function is performed. For example, a battery powered wireless monitor may be configured to sense intracardiac pressure periodically throughout the day without requiring a user to send a command to the wireless monitor each time it senses pressure. However, the battery may fail due to a fault, or after prolonged use, which may render the implantable device unusable. In some cases, the wireless implantable device may need to be configured to override its autonomous operation upon receiving wireless power or a wireless command from an external wireless device.

In some variations, a wireless implantable device may be configured to operate in a second mode, wherein the wireless implantable device may be configured to perform a task or a function in response to receiving wireless power and/or a wireless command from a user. For example, if the battery of an implantable wireless monitor is unusable (e.g., dead, fail), it may be configured to sense intracardiac pressure in response to receiving wireless power and/or a wireless command from a user (e.g., a patient). In some variations, such a second mode may comprise on-demand sensing (e.g., a wireless implantable device configured to sense a physiological parameter upon receiving wireless power and/or a wireless command from an external wireless device). In some variations, the wireless implantable device may be configured to operate in the second mode upon receiving wireless power and/or a wireless command from a user irrespective of the status of the battery. In some variations, the wireless power and/or the wireless command sent by a user may override the first mode of the wireless implantable device. For example, on-demand sensing may be performed by a physician during a checkup of the patient and/or by the patient when experiencing disease symptoms.

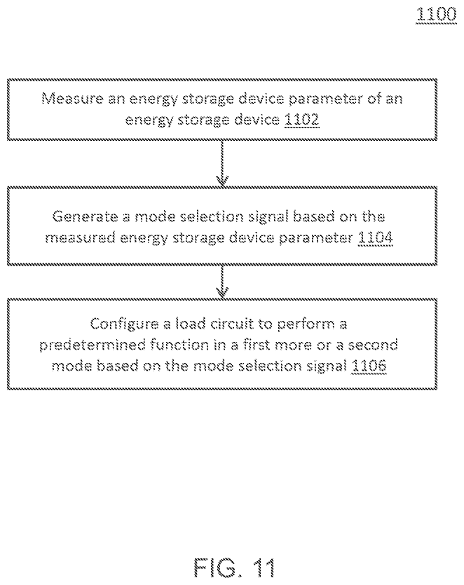

FIG. 11 is an illustrative flowchart of a method of operating a wireless implantable device. In some variations, the method (1100) may comprise the steps of measuring an energy storage device parameter of an energy storage device (1102), generating a mode selection signal based on the measured energy storage device parameter (1104), and configuring a load circuit to perform a predetermined function in a first mode or a second mode based on the mode selection signal (1106).

The energy storage device, the load circuit, and the predetermined function of the load circuit, as described herein, are applicable to any of the methods described herein. In some variations, the energy storage device may comprise one or more of a battery and a capacitor (e.g., which may comprise a super-capacitor). In some variations, the energy storage device may have a capacity of less than about 100 milli-Watthour. In some variations, the energy storage device parameter may comprise one or more of a battery voltage, a battery current, a battery impedance, a battery state of charge, a battery depth of discharge, a battery capacity, a battery energy, a battery power, a battery temperature, combinations thereof, and the like. In some variations, the energy storage device parameter may comprise one or more of a capacitor voltage, a capacitor current, a capacitor impedance (e.g., a capacitance, an equivalent series resistance or ESR of the capacitor, a resistance in parallel to the capacitance, and the like), a capacitor state of charge, a capacitor depth of discharge, a capacitor energy, a capacitor temperature, combinations thereof, and the like. In some variations, the energy storage device parameter may comprise a parameter of another circuit that may be used to infer a status of the energy storage device. For example, a resistor divider network may be connected to the battery voltage, and an output voltage of the resistor divider network may be measured in order to infer the battery voltage.

In some variations, the load circuit in the first mode may be configured to perform the predetermined function in an absence of a signal (e.g., wireless power, wireless data, wireless command, wireless signal, and the like) from an external device. In some variations, the load circuit in the second mode may be configured to perform the predetermined function in response to a signal (e.g., wireless power, wireless data, wireless command, wireless signal, and the like) from an external device. For example, the load circuit of a wireless implantable device in the first mode may be configured to monitor intracardiac pressure periodically throughout the day without the wireless implantable device requiring wireless power or a wireless command each time the intracardiac pressure is measured. Further, the load circuit in the second mode may be configured to measure intracardiac pressure and/or another physiological parameter upon receiving wireless power and/or a wireless command from an external wireless device.

In some variations, the predetermined function of the load circuit may comprise one or more of measuring a physiological parameter (e.g., intracardiac pressure, heart rate, and the like), measuring a parameter of the wireless implantable device (e.g., a structural parameter of the implantable device such as the thickness or motion of a prosthetic heart valve leaflet, tissue growth around an implantable device, scar tissue, and the like), controlling a wireless implantable device, delivering stimulation, and delivering therapy to a patient. In some variations, the physiological parameter may comprise one or more of an intracardiac pressure, an intravascular pressure, a blood pressure, a blood velocity, a blood flow, a blood oxygen level, a heart rate, a breathing rate, a temperature, a voltage, a current, an impedance, a neural signal, a heart sound, combinations thereof, and the like.

In some variations, the method (1100) may further comprise the steps of receiving wireless power using a transducer of the wireless implantable device, recovering at least a portion of the received wireless power using a power circuit of the wireless implantable device, and generating one or more supply voltages for the load circuit using a power detector circuit of the wireless implantable device. The transducer (e.g., ultrasonic transducer), the power circuit, the load circuit, and the power detector circuit, as described herein, are applicable to any of the methods described herein.

In some variations, the method (1100) may further comprise the step of measuring one or more of an energy storage device parameter and a power circuit parameter using the power detector circuit. The energy storage device parameter, as described herein, is applicable to any of the methods described herein. In some variations, the power circuit parameter may comprise one or more of a power circuit voltage, a power circuit current, a power circuit impedance, a power circuit power, a power circuit stored energy, a power circuit temperature, combinations thereof, and the like. For example, a power circuit parameter may comprise an output voltage of a rectifier. In some variations, the method (1100) may further comprise the step of storing energy in a capacitor of the wireless implantable device using the power circuit.

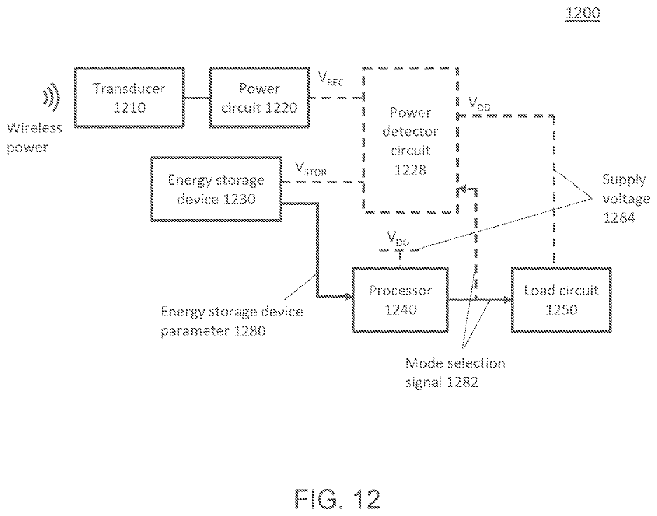

FIG. 12 depicts a wireless implantable device (1200) that may be configured for implementing the method (1100) of operating the wireless implantable device. The wireless implantable device (1200) may comprise an energy storage device (1230) configured to provide power to the wireless implantable device (1200), a load circuit (1250) coupled to the energy storage device (1230) and configured to perform a predetermined function, and a processor (1240) coupled to the energy storage device (1230). The processor (1240) may be configured to measure an energy storage device parameter (1280) and generate a mode selection signal (1282) based at least in part on the measured energy storage device parameter (1280). The load circuit (1250) may be configured to perform the predetermined function in a first mode or a second mode based on the mode selection signal (1282). The wireless implantable device, the energy storage device, the load circuit, the predetermined function, the processor, the energy storage device parameter, the first mode, and the second mode, as described herein, are applicable to any of the systems or methods described herein. In some variations, the processor (1240) may comprise one or more of a battery monitoring circuit configured to monitor a parameter of a battery (e.g., battery voltage, battery state of charge, and the like), a comparator configured to compare one or more parameters of the battery to one or more predetermined thresholds, and digital logic to generate the mode selection signal based upon the output of the comparator. For example, the processor (1240) may be configured to measure a battery voltage and generate a mode selection signal based on whether the battery voltage is greater or smaller than a threshold (e.g., 3 V). In some variations, if the battery voltage is above the threshold, then the mode selection signal may configure the load circuit to perform the predetermined function in the first mode (e.g., periodically monitoring a physiological parameter throughout the day); whereas, if the battery voltage is below the threshold (e.g., signifying that the battery is dead), then the mode selection signal may configure the load circuit to perform the predetermined function in the second mode (e.g., on-demand sensing). In some variations, the mode selection signal may be a digital signal comprising one or more digital bits.

In some variations, the wireless implantable device (1200) may further comprise a transducer (1210) configured to receive wireless power, a power circuit (1220) coupled to the transducer (1210) and configured to recover at least a portion of the received wireless power, and a power detector circuit (1228) coupled to the power circuit (1220) and the energy storage device (1230). The power detector circuit (1228) may be configured to generate one or more supply voltages (1284) for one or more of the load circuit (1250) and the processor (1240), as shown in FIG. 12. The transducer, the power circuit, and the power detector circuit, as described herein, are applicable to any of the systems described herein. In some variations, the power detector circuit (1228) may generate one or more supply voltages (1284) for one or more of the load circuit (1250) and the processor (1240) based upon the mode selection signal (1282) generated by the processor (1240). For example, the mode selection signal (1282) generated by the processor (1240) based on the measured battery voltage of less than about 3 V may control the power detector circuit (1228) to select the output of the power circuit (1220) for generating one or more supply voltages (1284) for the load circuit (1250). In some variations, one or more of the processor (1240), the load circuit (1250), and any sub-circuits of the processor (1240) and/or the load circuit (1250), may be powered by one or more of the energy storage device (1230), the output of the power circuit (1220) and the output of the power detector circuit (1228).

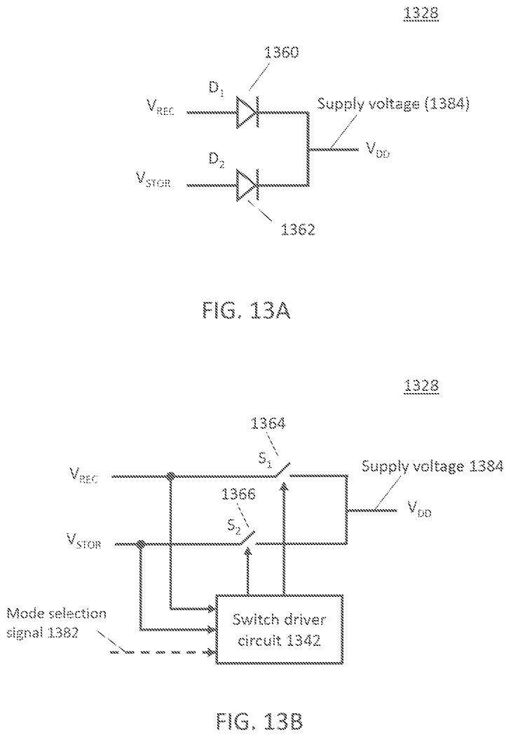

In some variations, the power detector circuit (1228) may comprise one or more of a power ORing circuit, a power combining circuit, a power selection circuit, one or more diodes and one or more switches. FIGS. 13A and 13B depict a power detector circuit (1328). In some variations, as shown in FIG. 13A, the power detector circuit (1328) may comprise diodes, D₁ (1360) and D₂ (1362), which may be configured to select the higher of the two input voltages, V_{REC} and V_{STOR}. Referring to FIG. 12, Vic may denote the output voltage of the power circuit (1220) and V_{STOR} may denote the voltage of the energy storage device (1230). In some variations, the diodes D₁ (1360) and D₂ (1362) may comprise one or more of a passive diode, a Schottky diode, an active diode, combinations thereof, and the like. In some variations, as shown in FIG. 13B, the power detector circuit (1328) may comprise switches, S₁ (1364) and S₂ (1366), which may be turned on or off for selecting which of the two input voltages, Vic or V_{STOR}, may be used to generate the output supply voltage, V_{DD} (1384). In some variations, the switches may be driven by a switch driver circuit (1342). In some variations, the switch driver circuit (1342) may comprise a comparator or other logic circuits that may be configured to compare V_{STOR} with Vic (e.g., to determine which of these voltages may be greater than the other) and/or to compare V_{STOR} or Vic to a reference voltage. In some variations, the switch driver circuit (1342) may comprise a low-power auxiliary path (e.g., comprising another power detector circuit such as the one depicted in FIG. 13A) to provide power to the comparator or other logic circuits comprising the switch driver circuit (1342). In some variations, the switch driver circuit (1342) may control the switches, S₁ (1364) and S₂ (1366), based on the mode selection signal (1382) generated by the processor.

Referring to FIG. 12, in some variations, the power detector circuit (1228) may be further configured to measure one or more of an energy storage device parameter, a power circuit parameter, combinations thereof, and the like. In some variations, the energy storage device parameter may comprise one or more of a battery voltage, a battery current, a battery impedance, a battery state of charge, a battery depth of discharge, a battery capacity, a battery energy, a battery power, a battery temperature, combinations thereof, and the like. In some variations, the energy storage device parameter may comprise one or more of a capacitor voltage, a capacitor current, a capacitor impedance (e.g., a capacitance, an equivalent series resistance or ESR of the capacitor, a resistance in parallel to the capacitance, and the like), a capacitor state of charge, a capacitor depth of discharge, a capacitor energy, a capacitor temperature, combinations thereof, and the like. In some variations, the power circuit parameter may comprise one or more of a power circuit voltage, a power circuit current, a power circuit impedance, a power circuit power, a power circuit stored energy, a power circuit temperature, combinations thereof, and the like.

In some variations, the wireless implantable device (1200) may further comprise one or more capacitors coupled to the power circuit (1220), wherein the power circuit (1220) may be configured to store energy in the one or more capacitors. In some variations, the processor (1240) may comprise one or more of an energy storage monitoring circuit, a battery monitoring circuit, a comparator, digital logic, combinations thereof, and the like. In some variations, the processor (1240) may be configured to be powered by one or more of the energy storage device (1230) and the power circuit (1220). In some variations, the transducer (1210) may comprise one or more acoustic (e.g., ultrasonic) transducers, and the wireless power received by the transducer (1210) may comprise acoustic (e.g., ultrasonic) power.

In some variations, a wireless monitor may be configured to operate in a first mode, such as a continuous monitoring mode. A user (e.g., a patient, a physician, etc.) may then use an external wireless device, which may be configured to send a wireless command to the wireless monitor via a downlink signal. Based on the command, the wireless monitor may start operating in a second mode, such as an on-demand sensing mode and/or a wireless uplink mode. For instance, in some variations, upon receiving the command, the wireless monitor may start transmitting sensed data stored in its memory to the external wireless device (i.e., wireless uplink mode). In some variations, upon receiving the command, the wireless monitor may take one or more samples of the physiological parameter and transmit raw or processed samples (e.g., after digitization and/or further digital signal processing) to the external wireless device via uplink signals.

In some variations, in the second mode, the external wireless device may send a power signal and/or a command to the wireless monitor, upon which the wireless monitor may use energy recovered from the power signal to take one or more samples of the physiological parameter, and send back the raw or processed samples to the external wireless device via uplink signals. In some variations, in the second mode, the external wireless device may only send a command to the wireless monitor, upon which the wireless monitor may use its stored energy (e.g., on a capacitor or a battery) to take one or more samples of the physiological parameter, and send back the raw or processed samples to the external wireless device via uplink signals. In some variations, on-demand sensing and/or wireless uplink from the wireless monitor may be performed during an imaging procedure (e.g., ultrasound imaging such as transthoracic echocardiography). For instance, on-demand sensing, wireless uplink and the imaging procedures may be time multiplexed.

D. Duty-Cycling Techniques for Energy-Efficient Implantable Devices

In some applications, wireless devices may comprise circuits such as wakeup receiver circuits which may be configured to detect any wireless signals sent by other wireless devices. For example, a wireless implantable device may comprise a wakeup receiver circuit to listen for any wireless signals (e.g., an interrogation signal or a wireless command) sent by an external wireless device, so that the wireless implantable device can perform an action in response (e.g., send a feedback signal to the external wireless device). In conventional wireless systems, a wakeup receiver circuit is always kept on because the time at which an external wireless device may send a signal to it may be unknown or unpredictable. However, keeping a wakeup receiver circuit always on may result in significant energy consumption, which may be a problem for miniature implantable wireless devices that have a constrained energy budget (e.g., are powered by a miniature battery). Further, in some applications, an implantable device may be powered by an energy storage device (e.g., a battery), and an energy storage monitoring circuit (e.g., a battery monitoring circuit) may be employed to monitor the energy state of the energy storage device for safe operation (e.g., to prevent over discharge of the energy storage device). In conventional systems, such an energy storage monitoring circuit is always kept on to enable continuous monitoring of the energy state of the energy storage device and avoid over discharge. However, this may also result in significant energy consumption, which may be undesirable for miniature implantable devices that have a constrained energy budget.

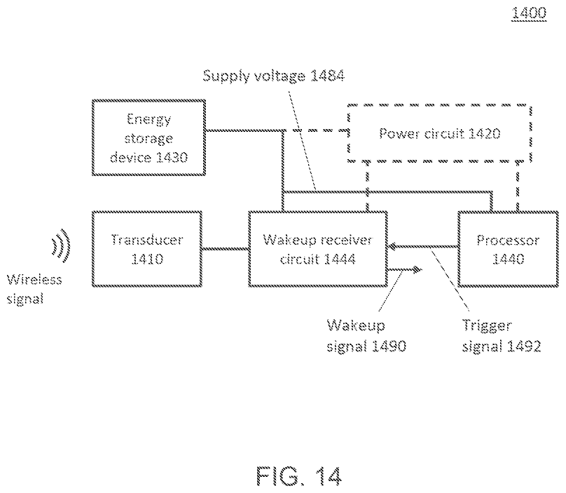

FIG. 14 depicts a wireless implantable device (1400) comprising a wakeup receiver circuit (1444). In some variations, the wireless implantable device (1400) may comprise a transducer (1410) configured to receive a wireless signal, a wakeup receiver circuit (1444) coupled to the transducer (1410), the wakeup receiver circuit (1444) configured to generate a wakeup signal (1490) in response to the wireless signal, a processor (1440) configured to generate a trigger signal (1492) for the wakeup receiver circuit (1444) based on a timer signal, and an energy storage device (1430) configured to provide power to the wakeup receiver circuit (1444) and the processor (1440). The wakeup receiver circuit (1444) may be configured to operate only upon receiving the trigger signal (1492). In some variations, the energy storage device (1430) may be configured to generate one or more supply voltages (1484) to power one or more of the wakeup receiver circuit (1444) and the processor (1440). The transducer, the wakeup receiver circuit, the processor, and the energy storage device, as described herein, are applicable to any of the systems or methods described herein.

In some variations, the processor (1440) of the wireless implantable device (1400) may comprise a timer circuit, wherein the timer circuit may be configured to generate the timer signal. For example, the timer circuit may comprise one or more of an oscillator circuit (e.g., a relaxation oscillator, an RC oscillator, a ring oscillator, and the like), a clock circuit, a counter circuit, digital logic, combinations thereof, and the like.

In some variations the trigger signal may comprise a periodic waveform having a predetermined repetition interval. In some variations, the predetermined repetition interval may be less than or equal to a duration of the wireless signal received by the transducer. For example, a wakeup receiver circuit may be periodically turned on by a processor of the wireless implantable device after a fixed or a variable time period denoted by T_{WUP}. In some variations, T_{WUP} may be pre-determined and programmed into the processor or a memory of the wireless implantable device (e.g., T_{WUP} may be set to 1 ms or 1 s, and the like). In some variations, T_{WUP} may be based on a duration of an interrogation signal sent by an external wireless device to the wireless implantable device. For example, the interrogation signal may be designed to last for a minimum duration denoted by T_{INT}. In such a system, if the wakeup receiver circuit of the wireless implantable device is turned on every T_{WUP}, where T_{WUP} is on the order of T_{INT} (e.g., T_{WUP} less than or equal to T_{INT}), then the wakeup receiver circuit may be very likely, or guaranteed, to be on at least for a short duration while the wireless implantable device is receiving the interrogation signal. This may allow the wireless implantable device to be able to detect an interrogation signal without having to keep its wakeup receiver circuit always on, thereby reducing energy dissipation of the wireless implantable device.

Referring to FIG. 14, in some variations, the wireless implantable device (1400) may further comprise a power circuit (1420) coupled to the energy storage device (1430) and configured to generate one or more supply voltages for powering one or more of the wakeup receiver circuit (1444) and the processor (1440). In some variations, the wireless signal received by the transducer (1410) of the wireless implantable device (1400) may be transmitted by a wireless device configured to be disposed physically separate from the wireless implantable device (e.g., located external to a patient). In some variations, the transducer (1410) may comprise one or more acoustic (e.g., ultrasonic) transducers, and the wireless signal received by the transducer (1410) may comprise an acoustic (e.g., ultrasonic) signal.

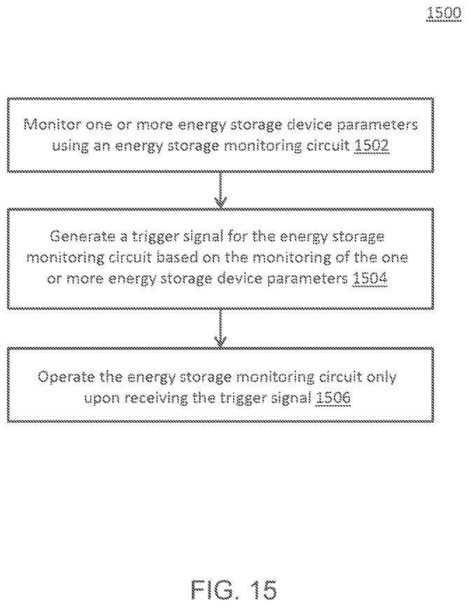

FIG. 15 is an illustrative flowchart of a method of operating an energy storage monitoring circuit. In some variations, the method (1500) may comprise the steps of monitoring one or more energy storage device parameters using an energy storage monitoring circuit (1502), generating a trigger signal for the energy storage monitoring circuit based on the monitoring of the one or more energy storage device parameters (1504), and operating the energy storage monitoring circuit only upon receiving the trigger signal (1506).

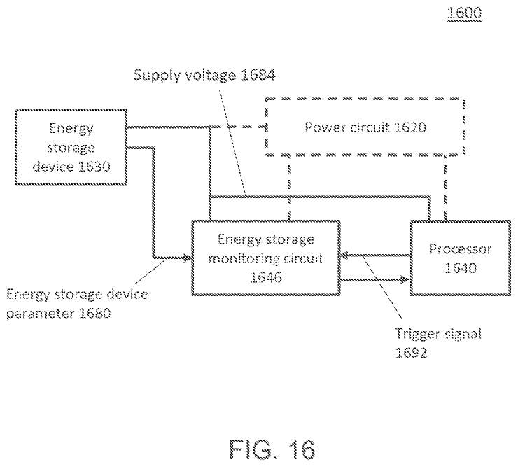

FIG. 16 depicts an implantable device (1600) comprising an energy storage monitoring circuit (1646). In some variations, an implantable device (1600) may comprise an energy storage device (1630) configured to provide power to the implantable device (1600), an energy storage monitoring circuit (1646) configured to monitor one or more energy storage device parameters (1680), and a processor (1640) configured to generate a trigger signal (1692) for the energy storage monitoring circuit (1646) based on the monitoring of the one or more energy storage device parameters (1680), wherein the energy storage monitoring circuit (1646) may be configured to operate only upon receiving the trigger signal (1692). The energy storage device, the energy storage monitoring circuit, the energy storage device parameter, and the processor, as described herein, are applicable to any of the systems or methods described herein.

In some variations, the processor (1640) may comprise a timer circuit. In some variations, the trigger signal may comprise a periodic waveform having a predetermined repetition interval. In some variations, the energy storage device parameter may comprise one or more of a battery voltage, a battery current, a battery impedance, a battery state of charge, a battery depth of discharge, a battery capacity, a battery energy, a battery power, a battery temperature, combinations thereof, and the like. In some variations, the energy storage device parameter may comprise one or more of a capacitor voltage, a capacitor current, a capacitor impedance (e.g., a capacitance, an equivalent series resistance or ESR of the capacitor, a resistance in parallel to the capacitance, and the like), a capacitor state of charge, a capacitor depth of discharge, a capacitor energy, a capacitor temperature, combinations thereof, and the like. In some variations, the energy storage device parameter may comprise a parameter of another circuit that may be used to infer a status of the energy storage device.

In some variations, the processor (1640) may be further configured to estimate one or more energy storage device parameters (1680). For example, a processor (1640) may be configured to predict a trend of a battery voltage (or state of charge) based upon one or more measurements of the battery voltage (or state of charge) across time, and/or using an estimation or measurement of the energy consumption rate of the implantable device (1600). In some variations, a processor (1640) may be configured to predict a trend of an energy storage device parameter (1680) based on information about one or more previous values of the energy storage device parameter (1680) and a discharge profile of the energy storage device (1630) or power consumption of the implantable device (1600). In some variations, the processor (1640) may be configured to generate the trigger signal (1692) based at least in part on the estimation of the one or more energy storage device parameters (1680). For example, a battery monitoring circuit may be configured to measure a battery voltage, and if the battery voltage is determined by a processor to be sufficiently above a threshold, then the processor may be configured to generate a trigger signal for turning on the battery monitoring circuit less frequently in order to conserve the battery's energy.

Referring to FIG. 16, in some variations, the wireless implantable device (1600) may further comprise a power circuit (1620) coupled to the energy storage device (1630) and configured to generate one or more supply voltages for powering one or more of the energy storage monitoring circuit (1646) and the processor (1640).

E. Monitoring Patient Activity in Conjunction with a Cardiovascular Parameter

In some applications, in addition to monitoring intracardiac or intravascular pressures using a wireless monitor, it may be important to monitor patient activity or other physiological parameters, including but not limited to one or more of a heart rate, a heart rate variability, a breathing rate or respiration, a thoracic impedance, a heart sound, a temperature, other pressures, a blood oxygen level, a blood glucose level, combinations thereof, and the like. This may be important to correlate pressure changes with a patient's activity level (e.g., rest, sleep, exercise, stress, and the like) or lifestyle, and/or to understand reasons for abnormal pressure changes. For instance, high pressures may be related to high activity levels such as exercise. Solutions are provided here to enable monitoring of patient activity in addition to, or in tandem with, intracardiac and/or intravascular pressure measurements.

In some variations, a wireless monitor may be configured to measure pressure, while an external device may be configured to measure patient activity or other physiological parameters. An external device for measuring patient activity or physiological parameters may include one or more of an external wireless device used for powering and communicating with the wireless monitor, a cell phone, a wearable device (e.g., a smart watch), a device carried by the patient, a device fastened to the patient's body (e.g., using a belt, an adhesive, etc.), another implantable device, combinations thereof, and the like. In some variations, a device for measuring patient activity may comprise one or more of an activity sensor, a motion sensor, an accelerometer, a micro-electromechanical system (MEMS) device, a force sensor, a pressure sensor, a temperature sensor, a sweat sensor, a heart rate sensor (e.g., electrocardiogram or ECG sensor, electrodes, etc.), a sensor to sense breathing rate, an audio sensor to detect heart sounds, combinations thereof, and the like. In some variations, the wireless monitor measuring pressure and the external device measuring patient activity, and/or the measurements performed by the wireless monitor and the external device, may be synchronized in time in order to accurately correlate the pressure data with the activity data. In some variations, such time synchronization may be performed by establishing a wireless link between an external wireless device and the wireless monitor.

In some variations, the wireless monitor may be configured to measure patient activity or other physiological parameters, in addition to pressure, wherein the wireless monitor may comprise one or more sensors as described above in the case of the external wireless device. In some variations, the wireless monitor may estimate patient activity based on an estimation of the heart rate from a pressure waveform. For instance, the time duration between two consecutive peaks or similar features of an intracardiac or an intravascular pressure waveform (e.g., duration between two consecutive systole or diastole phases) may represent the heart rate.

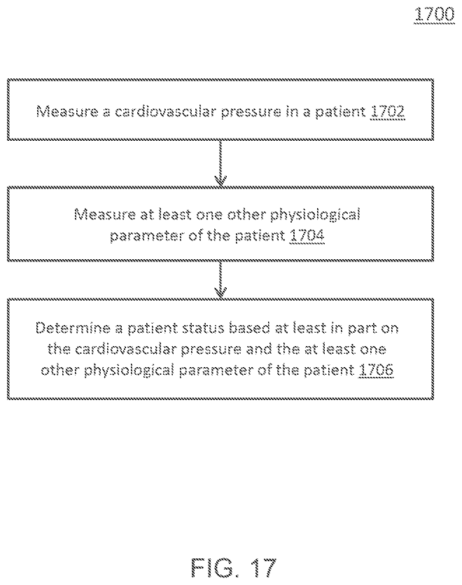

FIG. 17 is an illustrative flowchart of a method of cardiovascular pressure monitoring. In some variations, the method (1700) may comprise the steps of measuring a cardiovascular pressure in a patient (1702), measuring at least one other physiological parameter of the patient (1704), and determining a patient status based at least in part on the cardiovascular pressure and the at least one other physiological parameter of the patient (1706). In some variations, the method (1700) may further comprise synchronizing the measured cardiovascular pressure with the measurement of the other physiological parameter. In some variations, the synchronization may be performed by an external device configured to be disposed physically separate from the wireless monitor (e.g., located external to the patient). In some variations, the other physiological parameter may comprise one or more of a patient activity, a heart rate, a heart rate variability, a breathing rate, a thoracic impedance, a heart sound, a temperature, a blood pressure, a blood flow, a blood velocity, a blood oxygen level, a blood glucose level, combinations thereof, and the like.

In some variations, measuring the at least one other physiological parameter of the patient may be performed by an external device configured to be disposed physically separate from the wireless monitor (e.g., located external to the patient). In some variations, measuring the at least one other physiological parameter of the patient may be performed using a second wireless monitor implanted in the patient. In some variations, measuring the at least one other physiological parameter of the patient may be performed using the wireless monitor implanted in the patient.

In some variations, the method (1700) may further comprise digitizing the measured cardiovascular pressure using a processor of the wireless monitor. In some variations, the method (1700) may further comprise wirelessly transmitting digitized cardiovascular pressure from the wireless monitor to a wireless device configured to be disposed physically separate from the wireless monitor (e.g., located external to the patient). In some variations, the wireless device may comprise an external wireless device configured to be disposed physically separate from the wireless monitor (e.g., located external to the patient). In some variations, the wireless transmission may be performed using one or more of an acoustic signal, an ultrasonic signal, a radio-frequency signal, combinations thereof, and the like.

F. Powering an Implantable Device Using Energy Harvesting

In some variations, an implantable device may be powered using energy harvested from the motion of one or more bodily organs. For example, a wireless monitor implanted in or near the heart may be configured to harvest energy from the motion or pressure of the heart. Although typically such energy harvesting techniques provide low power densities compared to wirelessly powering a wireless monitor from an external wireless device, it may be sufficient to support one or more functions of the wireless monitor that require low energy. Additionally, since such energy sources are available throughout the day, a wireless monitor using such energy harvesting techniques may be able to accumulate sufficient energy over time to support one or more of its functions.

In some variations, a wireless monitor may comprise an ultrasound or an acoustic transducer (e.g., a piezoelectric transducer) to harvest energy from the surrounding pressure and/or organ motion. In some variations, this ultrasound or acoustic transducer may be the same transducer used for exchanging (e.g., receiving, transmitting) wireless signals (e.g., power, data, commands, etc.) with an external wireless device. In some variations, a multiplexer circuit may be used to decouple these different functions of the transducer. In some variations, the multiplexer circuit may be configured to always keep the transducer connected to one or more power circuits for energy harvesting.

In some variations, a wireless monitor may comprise a pressure or a force transducer to harvest energy from the surrounding pressure and/or organ motion. In some variations, the same or a different pressure transducer may be used to sense blood pressure for the purpose of monitoring a patient's disease. In some variations, the wireless monitor may additionally comprise an ultrasound and/or an RF transducer for uplink and/or downlink data communication with an external wireless device. In some variations, the ultrasound and/or RF transducer may be additionally configured to also receive wireless power from the external wireless device.

G. Exchanging Wireless Signals and Measuring Pressure Using a Single Transducer of a Wireless Monitor

In some variations, a wireless monitor may comprise a single transducer (e.g., a piezoelectric transducer) for measuring pressure as well as performing wireless functions such as receiving wireless power and/or downlink signals from an external wireless device, and/or transmitting uplink signals to an external wireless device. The voltage across the terminals of the transducer may be indicative of the surrounding pressure. A multiplexer circuit may be used to decouple these different functions of the single transducer. Such a configuration of the wireless monitor may be advantageous for its miniaturization.

Although the foregoing variations have, for the purposes of clarity and understanding, been described in some detail by illustration and example, it will be apparent that certain changes and modifications may be practiced, and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

1. A wireless monitor, comprising:
a sensor configured to measure a physiological parameter of a patient at a first resolution; and
a processor configured to generate physiological parameter data based on the measured physiological parameter of the patient at the first resolution,
wherein the sensor is configured to measure the physiological parameter of the patient at a second resolution based at least in part on the physiological parameter data.

2. The wireless monitor of claim 1, wherein the first resolution and second resolution comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, and a filter cut-off frequency.

3. (canceled)

4. The wireless monitor of claim 1, wherein measuring the physiological parameter at the first resolution consumes lower energy than measuring the physiological parameter at the second resolution.

5. The wireless monitor of claim 1, wherein the wireless monitor is implanted in a patient.

6. (canceled)

7. The wireless monitor of claim 1, wherein the physiological parameter comprises one or more of an intracardiac pressure, an intravascular pressure, a blood pressure, a blood velocity, a blood flow, a blood oxygen level, a heart rate, a breathing rate, a temperature, a voltage, a current, an impedance, a neural signal, and a heart sound.

8. (canceled)

9. (canceled)

10. The wireless monitor of claim 1, further comprising a memory configured to store one or more of the measured physiological parameter at the first resolution, the measured physiological parameter at the second resolution, the physiological parameter data, and resolution data.

11. The wireless monitor of claim 1, further comprising a wireless transmitter configured to wirelessly transmit one or more of the measured physiological parameter at the first resolution, the measured physiological parameter at the second resolution, the physiological parameter data, and resolution data to a wireless device.

12. The wireless monitor of claim 11, wherein the wireless device is an external wireless device configured to be disposed physically separate from the wireless monitor.

13. (canceled)

14. The wireless monitor of claim 1, wherein the sensor comprises a resolution setting of the sensor.

15. The wireless monitor of claim 14, wherein the processor is configured to adjust the resolution setting of the sensor based at least in part on the physiological parameter data.

16. The wireless monitor of claim 14, wherein the resolution setting comprises one or more of a number of bits per sample, a voltage, a current, a sampling rate, a sampling duration, a number of samples, an over-sampling ratio (OSR), a frequency, a phase, an impedance, and a filter cut-off frequency.

17. The wireless monitor of claim 14, further comprising a memory configured to store the resolution setting of the sensor.

18. The wireless monitor of claim 14, further comprising a wireless transmitter configured to wirelessly transmit the resolution setting of the sensor to a wireless device.

19. (canceled)

20. A method of monitoring a patient, comprising:
measuring a physiological parameter of the patient at a first resolution using a wireless monitor;
generating physiological parameter data based on the measured physiological parameter at the first resolution;
measuring the physiological parameter of the patient at a second resolution using the wireless monitor based at least in part on the physiological parameter data; and
estimating a physiological state of the patient based at least in part on the measured physiological parameter at the second resolution.

21. The method of claim 20, wherein the first resolution and the second resolution comprise one or more of an amplitude of the physiological parameter, a timing of the physiological parameter, a number of bits per sample, a voltage, a current, a sampling rate, a sampling duration, a number of samples, an over-sampling ratio (OSR), a frequency, a phase, an impedance, and a filter cut-off frequency.

22. (canceled)

23. (canceled)

24. (canceled)

25. (canceled)

26. The method of claim 20, wherein the physiological parameter comprises one or more of an intracardiac pressure, an intravascular pressure, a blood pressure, a blood velocity, a blood flow, a blood oxygen level, a heart rate, a breathing rate, a temperature, a voltage, a current, an impedance, a neural signal, and a heart sound.

27. (canceled)

28. (canceled)

29. (canceled)

30. The method of claim 20, further comprising detecting a physiological event based at least in part on one or more of the measured physiological parameter at the first resolution and the physiological parameter data.

31. The method of claim 30, wherein the detected physiological event comprises one or more of arrhythmia, atrial fibrillation, ventricular tachycardia, sleep apnea, abnormally high blood pressure, abnormally low blood pressure, abnormal pressure variation, abnormal heart rate, and abnormal heart rate variability.

32. The method of claim 20, further comprising storing one or more of the measured physiological parameter at the first resolution, the measured physiological parameter at the second resolution, the physiological parameter data, and resolution data in a memory of the wireless monitor.

33. The method of claim 20, further comprising wirelessly transmitting one or more of the measured physiological parameter at the first resolution, the measured physiological parameter at the second resolution, the physiological parameter data, and resolution data from the wireless monitor to a wireless device.

34.-126. (canceled)
